(19) United States
(12) Patent Application Publication
(10) Pub. No.: US 2007/0134699 A1
(43) Pub. Date: Jun. 14, 2007

(54) Nano-scale ligand arrays on substrates for particle beam instruments and related methods

(75) Inventors: William Roy Glover III, North Reading, MA (US); Scott A. McCallister, Bryn Mawr, PA (US)

Correspondence Address: WOLF GREENFIELD & SACKS, P.C., 600 ATLANTIC AVENUE, BOSTON, MA, 02210-2206, US

(73) Assignee: ZS Genetics, Inc., Bryn Mawr, PA (US)

(21) Appl. No.: 11/595,819

(22) Filed: Nov. 9, 2006

Related U.S. Application Data
(60) Provisional application No. 60/734,954, filed on Nov. 9, 2005.
(60) Provisional application No. 60/834,205, filed on Jul. 28, 2006.

(51) Int. Cl. C12Q 1/68; C12M 3/00

(52) U.S. Cl. 435/6; 435/287.2; 977/924

(57) ABSTRACT

Substrates and arrays that can be used for biological analysis with particle beam instruments are provided. In one embodiment, a substrate for an array is constructed and arranged to be used for imaging samples with a particle beam instrument such as a transmission electron microscope. The substrate can include one or more ligands (e.g., nucleic acids, polypeptides, oligosaccharides, and synthetic polymers) which may form an array. Corresponding changes in labeling chemistry can allow for ligands, binding partners and other relevant materials to be identifiable, quantitatable, and even sequenceable via modified forms of electron microscopy. In certain embodiments, the array dimensions are on the order of nanometers per functional region rather than micrometers as in certain conventional arrays. With these dimensions, smaller amounts of sample material can be used and more accurate genetic analyses performed. These smaller substrate dimensions may also give rise to dramatically reduced production costs, amongst other advantages. The transparency of the substrate, due to thinness, material type and other factors, may provide a suitable contrast ratio of the labeled molecules against the substrate that result in higher quality readings and lower cost analysis than some conventional techniques.

### RELATED APPLICATIONS

This application claims priority under 35 U.S.C. §119(e) to U.S. Provisional Patent Application No. 60/734,954, filed Nov. 9, 2005, and entitled “Photolithographic, Electro-Lithographic, and Other Means for Manufacturing Nano-Scale Polymer Arrays”, and U.S. Provisional Patent Application No. 60/834,205, filed Jul. 28, 2006, and entitled “Manufacturing Methods and Devices for Nano-Scale Ligand Arrays on Substrates for Particle Beam Instruments”, both of which are incorporated herein by reference.

### FIELD OF THE INVENTION

The present invention relates generally to substrates, arrays and methods associated therewith, and more specifically, to substrates and arrays that can be used for biological analysis with particle beam instruments.

### BACKGROUND OF THE INVENTION

The relationship between structure and function of macromolecules is of fundamental importance in the understanding of biological systems. This relationship is important to understanding, for example, the fictions of enzymes, structural proteins, and signaling proteins, ways in which cells communicate with each other, as well as mechanisms of cellular control and metabolic feedback.

The human genome project was directed toward determining the complete sequence of the genome of the human organism. Although this sequence does not correspond to the sequence of any specific individual, it provides significant information as to the general organization and specific sequences contained within segments from particular individuals. It also provides mapping information which is useful for further detailed studies. To complete the sequencing of a human genome would require the determination of approximately 3.2 billion base pairs. The need for rapid, accurate, and inexpensive sequencing technology is apparent in such an endeavor.

Conventional procedures used for sequencing include the Sanger dideoxy method, see, e.g., Sanger et al. (1977) Proc. Natl. Acad. Sci. USA, 74:5463-5467, and the Maxam and Gilbert method, see, e.g., Maxam et al., (1980) Methods in Enzymology, 65:499-559. The Sanger method utilizes enzymatic elongation procedures with chain terminating nucleotides. The Maxam and Gilbert method uses chemical reactions exhibiting specificity of reaction to generate nucleotide specific cleavages. Both methods typically involve a practitioner performing a large number of complex manual manipulations. These manipulations can require isolating homogeneous DNA fragments, elaborate and tedious preparing of samples, preparing a separating gel, applying samples to the gel, electrophoresing the samples into this gel, working up the finished gel, and analyzing the results of the procedure.

Procedures used for gene expression are generally based on immobilizing mRNA or cDNA (prepared via reverse transcriptase PCR from MRNA) to microarrays, and estimating quantity from fluorescent images. Some of these procedures are described in U.S. Pat. Nos. 5,405,783; 5,424,186; 5,445,934; 5,744,305; 6,261,776; 6,406,844; 6,416,952; 6,506,558; and 5,143,854. These procedures may introduce substantial error via high levels of amplification and intrinsic limits to fluorescent imaging. Consequently, typically only relative (rather than absolute) quantities of molecules are identified, and the quantification is subject to substantial error, especially at low levels of expression. For these reasons, current gene expression experiments with microarrays are somewhat intrinsically imprecise and are often performed two or more times for even rough accuracy.

Thus, less expensive, more reliable and labor efficient means for analyzing biological macromolecules are needed. Reductions in cost and increases in speed would be advantageous. Additionally, smaller sample amounts that can be processed on a smaller size array which would save scarce or expensive sample materials would be beneficial.

### SUMMARY OF THE INVENTION

Substrates and arrays that can be used with particle beam instruments are provided.

In one embodiment, a substrate is provided. The substrate comprises at least a first imaging window having a thickness of less than or equal to 30 nanometers, and at least a first linker attached to a surface of the first imaging window.

In another embodiment, a substrate comprises at least a first imaging window, and at least a first ligand attached to a surface of the first imaging window. The first imaging window may be capable of producing a contrast ratio of greater than 1.05:1 upon exposure of the first imaging window to a particle beam and upon imaging of the first ligand.

In another embodiment, a substrate comprises a first portion comprising a silicon oxide-based layer and a silicon nitride-based layer, and a second portion having a thickness greater than the first portion. The second portion may include a silicon oxide-based layer, a silicon nitride-based layer, and a Si layer. A first linker may be attached to a surface of the first portion of the substrate.

In another embodiment, a substrate comprises at least a first imaging window having a thickness of less than or equal to 30 nanometers, and at least a first ligand attached to a surface of the first imaging window.

Unless defined otherwise, all technical and scientific terms used herein have the same meaning as commonly understood by one of ordinary skill in the art to which this invention belongs. Certain terms are defined below for the sake of clarity and ease of reference.

A “ligand” is a molecule that can be recognized by a particular binding partner. Examples of ligands that can be investigated by the present invention include, but are not restricted to, nucleic acid polymers (which may be single-or double-stranded), agonists and antagonists for cell membrane binding partners, toxins and venoms, viral epitopes, hormones (e.g., steroids, etc.), hormone binding partners, peptides, enzymes, enzyme substrates, cofactors, drugs, (e.g., opiates, etc), lectins, sugars, oligonucleotides, nucleic acids, nucleic acid analogues, nucleic acids modified for improved reactivity or label effectiveness, oligosaccharides, proteins, oligopeptides, antigens, antibodies (e.g., monoclonal antibodies), and combinations thereof. Specific examples of ligands are described in U.S. Pat. Nos. 5,405,783; 5,424,186; 5,445,934; 5,744,305; 6,261,776; 6,406,844; 6,416,952; and 6,506,558, which are incorporated herein by reference. In some instances, certain ligands (e.g., avidin, proteins, DNA strands) can have more than one active site such that more than one binding partner can be bound to the ligand. The bound binding partner(s) may also include more than one active site, and, in some cases, can act as a ligand for another binding partner. A ligand-binding partner pair can itself act as a ligand for subsequent binding partner interactions.

A “binding partner” is a molecule that has an affinity for a given ligand. Binding partners may be naturally-occurring or manmade molecules. Also, they can be employed in their unaltered state or as aggregates with other species. Binding partners may be attached, covalently or noncovalently, to a ligand, either directly or via a specific binding substance. Examples of binding partners which can be employed by the present invention include, but are not restricted to, antigens, antibodies, cell membrane binding partners, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Binding partners are sometimes referred to in the art as “anti-ligands”, “receptors”, or “targets” (e.g., a “target polynucleotide sequence”). As these terms are used herein, no difference in meaning between such terms is intended. A “ligand-binding partner pair” is formed when two macromolecules have combined through molecular recognition to form a combined entity. In many embodiments, certain binding partners are isolated from samples (e.g., detected).

A “monomer” is a member of the set of small molecules which are, or can be, joined together to form a polymer. The set of monomers includes, but is not restricted to, for example, the set of common L-amino acids, the set of D-amino acids, the set of synthetic and/or natural amino acids, the set of nucleotides, including standard and modified versions of nucleotides, and the set of pentoses and hexoses. The particular ordering of monomers within a polymer is referred to herein as the “sequence” of the polymer. As used herein, monomer can also refer to any member of a basis set for synthesis of a polymer. For example, dimers of the 20 naturally occurring L-amino acids form a basis set of 400 monomers for synthesis of polypeptides. Different basic sets of monomers may be used at successive steps in the synthesis of a polymer. Furthermore, each of the sets may include protected members which are modified after synthesis. The invention is described herein primarily with regard to the preparation of molecules containing sequences of monomers such as amino acids, but could readily be applied in the preparation of other polymers. Such polymers include, for example, both linear and cyclic polymers of nucleic acids, polysaccharides, phospholipids, and peptides having either alpha-, beta-, or gamma-amino acids, heteropolymers in which a known drug is covalently bound to any of the above, polynucleotides, polyurethanes, polyesters, polycarbonates, polyureas, polyamides, polyethyleneimines, polyarylene sulfides, polysiloxanes, polyimides, polyacetates, or other polymers which will be apparent upon review of this disclosure. Such polymers are “diverse” when polymers having different monomer sequences are formed at different predefined regions of a substrate.

A “feature” is a localized area on a substrate surface that can include one or more ligands. In some embodiments, all ligands in a feature are identical (e.g., they may have the same composition of monomers). In other embodiments, however, a single feature may include different ligands. For example, several types of ligands could intentionally be included in a single feature so as to provide an initial screening for biological activity, after which materials within these features exhibiting significant binding can be further evaluated. In another embodiment, a feature may include molecules or other components (in addition to or excluding ligands), such as a focusing aid for a particle beam (e.g., for the purpose of improving cost and/or quality of the application). A feature may have any convenient shape, e.g., circular, rectangular, elliptical, wedge-shaped, etc. Different features may have different shapes, different sizes and/or different numbers of ligands.

An “array” comprises one or multiple features on a substrate surface where a sample containing binding partners can be applied. The features may be positioned in any convenient shape or pattern including, but not limited to, a rectangular matrix, a matrix with offset rows, or radial lines from a central point. Different arrays may have different shapes, numbers of features or distance between features. In certain embodiments where a single substrate has multiple arrays, a mechanism may be applied to prevent cross-contamination between different samples applied to the different arrays.

A “linker” can include a molecule (e.g., a series of monomers) that can be functionalized at an end and/or at any point(s) in between. In one embodiment, a linker is designed to attach to the substrate surface at one end, covalently or non-covalently, and attach to ligands at the other end using common chemistry methods. In another embodiment, a ligand attaches to a non-end portion of the linker. In yet another embodiment, a ligand attaches to more than one portion of a linker. The linker molecules may be, for example, aryl acetylene, ethylene glycol oligomers including from 2-10 monomers or more, diamines, diacids, amino acids, or combinations thereof, and may include any suitable functional group (e.g., amino, aldehyde, etc.). The number of molecules or monomers in a linker can vary by application.

“Lithographic assembly methods” are methods that use lithography to activate (e.g., irradiate) a precisely defined area or multiple areas on a surface with an exposure as a component of a process to perform biochemical synthesis and/or assembly of molecules (e.g., to form a feature). Masks or maskless techniques to direct the activation can be used to perform the lithography steps. Types of activation include, but are not limited to, light, electron beam, electrical currents or other spatially localized activators. Combinations of activation types may be used. Lithographic assembly methods also including spotting of complete ligands to form features. Lithographic assembly methods are familiar to those of ordinary skill in the art.

“Lithographic shaping methods” are methods that use lithography to activate a precisely defined area or multiple areas on a surface with one exposure as a component of a process to alter the shape of a substrate, wafer or other material. The other process components may use chemical or other substances to change or shape the material in areas where the lithography has activated or deactivated a material with a protective method. Masks or maskless techniques to direct the activation can be used to perform the lithography steps. Types of activation include, but are not limited to, light, electron beam, electrical currents or other spatially localized activators. Combinations of activation types may be used. Lithographic shaping methods are familiar to those of ordinary skill in the art.

“Protective methods” are methods for protecting ligands from becoming chemically active. The methods may involve the use of components that can be selectively removed. As an example, photolithographic assembly methods may use a photo-resist material to keep ligands from having an active chemical reaction with other materials unless a selective area is exposed to a light source which allows the resist to be washed away thereby exposing the ligand. Another example is a species that is bound to one or more monomer units to prevent polymerization and which may be selectively removed therefrom to expose an active site such as, in the specific example of an amino acid, an amine group. The species can be removed by a specific process, examples of which include, but are not limited to, light exposure, electron beam exposure or local exposure to acidic conditions. In the example of light exposure, the protective method is commonly called a photo-labile protecting group. Such techniques are well known to those of skill in the art.

A “particle beam instrument” is an instrument that uses particle beams for microscopy at the nano-scale or sub-nanometer scale. In some embodiments, binding on arrays described herein can be detected using transmission electron microscopes. In other embodiments, binding on arrays described herein can be detected using scanning electron microscopes, sub-150 nanometer photon microscopes, and other particle beam instruments, especially charged particle beams.

A “detector instrument” is a device or multiple devices used to collect the particle beam species after the beam has interacted with an array and substrate and turn them into digital images for storage and subsequent analysis. CCD cameras imaging a phosphor plate hit by the particle beam, CMOS cameras and other imaging and data collection systems may be used.

A “data analysis module” is a system comprising a computer, storage devices, software and other components to receive and analyze signals from the detector instrument. The analyses will translate the detector instrument signals into information outputs specific to the application in operation.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows a top view of a portion of a substrate including a plurality of imaging windows according to an embodiment of the invention;

FIG. 1B shows a cross-section of a portion of the substrate of FIG. 1A according to an embodiment of the invention;

FIG. 1C is a magnified view of the substrate of FIG. 1B including a plurality of probes, ligands, and linkers according to an embodiment of the invention;

FIG. 2 shows a cross-section of a portion of a substrate including a plurality of layers according to an embodiment of the invention;

FIG. 3A shows a cross-section of a portion of substrate including an imaging window in the form of a void according to an embodiment of the invention;

FIG. 3B shows a top view of the substrate of FIG. 3A according to an embodiment of the invention;

FIG. 4 shows a top view of a portion of a substrate including imaging windows having a high aspect ratio according to an embodiment of the invention;

FIG. 5 shows one imaging window of the substrate of FIG. 4 with multiple ligand-binding partner pairs according to an embodiment of the invention;

FIGS. 6A and 6B show top and cross-sectional views, respectively, of a wafer according to an embodiment of the invention;

FIG. 7 shows different wafer sections which correspond to multiple substrates according to an embodiment of the invention;

FIGS. 8A-8C show top, side, and back views of a substrate including a plurality of frames according to an embodiment of the invention;

FIG. 9 shows a feature including a plurality of unhybridized probes according to an embodiment of the invention;

FIG. 10 shows hybridized probes of a feature according to an embodiment of the invention;

FIG. 11A shows a feature including a relatively low density of ligand-binding partner pairs before being subjected to a directional molecular alignment technique according to an embodiment of the invention;

FIG. 11B shows the feature of FIG. 11A after being subjected to a directional molecular alignment technique according to an embodiment of the invention; and

FIG. 12 shows a feature including a relatively high density of ligand-binding partner pairs according to an embodiment of the invention.

### DETAILED DESCRIPTION

One aspect of the invention provides a substrate having a combination of materials and dimensions that allows the substrate to have distinct physical properties. Specifically, in one embodiment, the materials and dimensions of the substrate allow it to be used for imaging samples with a particle beam instrument such as a transmission electron microscope. The substrate can include one or more ligands (e.g., nucleic acids, polypeptides, oligosaccharides, and synthetic polymers) which may form an array. Corresponding changes in labeling chemistry can allow for ligands, binding partners and other relevant materials to be identifiable, quantitatable, and even sequenceable via modified forms of electron microscopy. In certain embodiments, the array dimensions are on the order of nanometers per functional region rather than micrometers as in certain conventional arrays. With these dimensions, smaller amounts of sample material can be used and more accurate genetic analyses performed. These smaller substrate dimensions may also give rise to dramatically reduced production costs, amongst other advantages. The transparency of the substrate, due to thinness, material type and other factors, may provide a suitable contrast ratio between the labeled molecules and the substrate that result in higher quality readings and lower cost analysis than some conventional techniques.

As discussed in more detail below, embodiments of the invention may be used for identification, quantification, sequencing, fingerprinting, and mapping of polymers, particularly biological polymers. Various embodiments of the invention may be applied, for example, in the sequencing, fingerprinting, identification, quantification, or mapping of nucleic acids, polypeptides, oligosaccharides, and synthetic polymers.

Aspects of the present invention may be combined with the description of certain embodiments in U.S. Patent Publication Nos. 2006/0024716, 2006/0024717, 2006/0024718, and 2006/0029957 all entitled, “Systems and Methods of Analyzing Nucleic Acid Polymers and Related Components”, each of which is incorporated herein by reference in its entirety. These references may provide, for example, methods and devices for incorporating contrast atom labels in a biologic sample that are designed to interfere with a beam from a particle beam instrument. In certain embodiments, the labeled sample materials are binding partners, which can be bound to ligands in an array on a suitable substrate. A particle beam may be directed through the array and the labels can create interference patterns that are then read by a detector instrument and processed by a data analysis module.

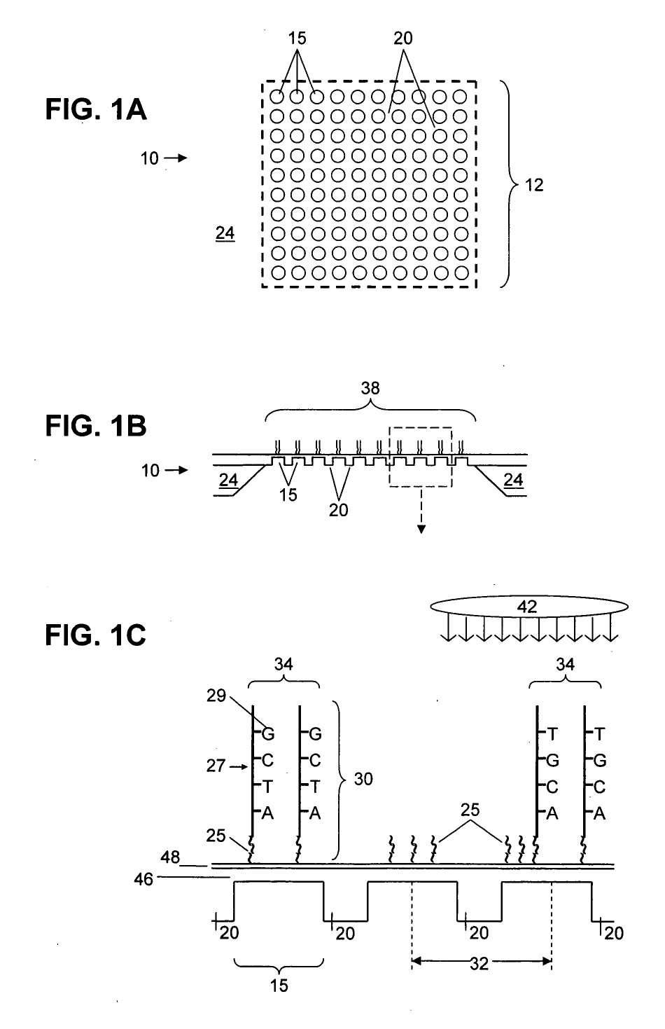

FIG. 1A shows a top view of a portion of a substrate according to one embodiment of the invention. In the illustrative embodiment of FIG. 1A, substrate 10 includes a frame 12 including a plurality of imaging windows 15, which can be used for determining (e.g., imaging) a species attached thereto. Each of the imaging windows are separated from one another by at least one divider portion 20. As shown in the illustrative embodiment of FIG. 1B, a side view of substrate 10, and FIG. 1C, a magnified view of a portion of FIG. 1B, imaging windows 15 can include linkers 25 attached to a surface of the imaging window. The linker may have attached thereto a ligand 27 such as a nucleic acid polymer, which may be formed of one or more monomers 29. The linker and ligand together form a probe 30, which may allow binding of a binding partner from a sample. In some instances, multiple probes (e.g., having the same composition of monomers) are attached to a surface of an imaging window to form a feature 34. One or more feature(s) 34 form an array 38, which may be contacted by the same sample. The array may include one feature, or multiple features, per imaging window, and optionally several imaging windows having features attached thereto for detecting a binding partner from a sample.

As shown in FIG. 1C, imaging windows 15 can include one or more linkers 25, ligands 27, probes 30, features 34, and/or a combination thereof attached to a surface of the imaging windows. Accordingly, although the primary description herein involves substrates having features and probes attached thereto, as well as substrates having a plurality of imaging windows, it should be understood that the substrates of the invention can also have other arrangements. For example, in some embodiments, a substrate may include only linkers or only ligands attached thereto instead of probes. In another embodiment, a substrate may include only one imaging window instead of a plurality of imaging windows and only one feature (e.g., for detecting a single species in a sample having many species). In yet another embodiment, a substrate may have at least one imaging window having a thickness of less than or equal to 30 nanometers, and at least one linker attached to a surface of the imaging window. In yet another embodiment, a substrate can include more than one array, each of which can be contacted with a different sample. Also, shown in FIG. 1C, probes 30 may be positioned substantially perpendicular to a surface of imaging window 15. It should be understood, however, that probes can be positioned in any suitable orientation on a surface of an imaging window. For example, in some embodiments, probes may lie substantially horizontally across a surface of an imaging window. As described in more detail below, other configurations and arrangements with respect to imaging windows, linkers, ligands, probes, features, arrays, and frames on a substrate are also possible.

As discussed in more detail below, in some embodiments, binding of a binding partner from a sample to probes of an array can be determined by a particle beam instrument. As such, imaging windows 15 may be constructed and arranged such that interference (e.g., noise) is minimized when used with a particle beam instrument. For example, as shown in the exemplary embodiment illustrated in FIG. 1B, imaging windows 15 may form the thinnest portion of a substrate and divider portions 20 may separate adjacent imaging windows. As shown, divider portions 20 have a thickness greater than that of imaging windows 15, however, in other embodiments, the divider portions may have the same thickness as that of the imaging windows. In one particular embodiment, imaging windows 15 have a thickness of less than or equal to 30 nanometers, divider portions 20 have a thickness of greater than 30 nanometers (e.g., between 31-200 nanometers), and frame 12 may be supported by support portions 24 of the substrate, which may have a thickness of, for example, greater than 200 microns. It should be understood that such values of thicknesses of imaging windows, divider portions, and support portions of a substrate are exemplary and that other dimensions, configurations and arrangements are possible, and may depend on the particular application (e.g., gene expression vs. sequencing). For instance, in one embodiment, imaging window 15 has a thickness such that when a ligand attached to a surface of the imaging window is subsequently exposed to particle beam 42 and imaged, a contrast ratio of greater than 1.1:1 is produced. The contrast ratio is defined as a measure of a signal (e.g., intensity or degree of darkness or lightness) produced by a label in the sample (e.g., a contrast atom label) when exposed to a particle beam, compared to a signal produced by the background (e.g., substrate and other materials, if present, in the same imaging area) as measured by a particular instrument. For example, a contrast ratio of 1.1:1 means that the signal produced by the label is 10% above or below that of the background. In some cases where a labeled ligand-binding partner pair is attached to a substrate and imaged by a transmission electron microscope (TEM), a contrast ratio of 1.1:1 means that the label is 10% darker than that of the substrate on an image produced by such a method.

Although FIG. 1C shows particle beam 42 being directed towards the front side of, and perpendicular to, the substrate, it should be understood that the particle beam may be directed at any suitable angle and/or towards the back side of the substrate in other embodiments.

As used herein, the term “determining” generally refers to the analysis of a species or signal, for example, quantitatively or qualitatively, and/or the detection of the presence or absence of the species or signals. “Determining” may also refer to the analysis of an interaction between two or more species or signals, for example, quantitatively or qualitatively, and/or by detecting the presence or absence of the interaction.

Advantageously, the configurations and arrangements of features on substrates of the invention may be much smaller than those of some microarrays, which can result in reduced requirements for sample materials and lower cost. For example, features of substrate 10 may span an area of less than or equal to 35 microns, less than or equal to 10 square microns, less than or equal to 5 square microns, less than or equal to 3 square microns, less than or equal to 1 square micron, less than or equal to 500 square nanometers, or less than or equal to 300 square nanometers. In addition, the window pitch between first and second imaging windows defined by length 32 (FIG. 1C) may be less than or equal to 10 microns, less than or equal to 5 microns, less than or equal to 3 microns, or less than or equal to 1 micron, less than or equal to 500 nm, less than or equal to 100 nm, less than or equal to 50 nm, or less than or equal to 25 nm. Similarly, due to the high sensitivity of the methods of detection described herein, each feature of substrate 10 can be made up of a fewer number of probes (or ligands). For instance, in certain embodiments, a feature may have less than 10⁵, less than 10⁴, less than 10³, less than 500, less than 200, or less than 100, less than 1, or less than 0.1, or less than 0.02 probes (or ligands) per square micron. The decrease in the size of each feature of substrates described herein, and the decrease in feature pitch of adjacent features, can allow a greater density of features on the substrate. For example, substrates of the invention may include greater than or equal to 10⁶, 10⁸, or 10¹⁰ features per square millimeter. In some cases, however, a substrate can include fewer numbers of features (e.g., 1-10⁶ features per square millimeter). Moreover, because a single imaging window may include more than one feature (e.g., greater than 5, greater than 10, greater than 20, greater than 50, greater than 100, or greater than 500 features per imaging window), the size of the array can be substantially reduced. For example, certain arrays described herein may have an area of less than or equal to 10 square millimeters, less than or equal to 7 square millimeters, less than or equal to 5 square millimeters, less than or equal to 3.5 square millimeters, less than or equal to 100 square microns, less than or equal to 10 square microns, less than or equal to 1 square micron, or less than or equal to 0.2 square microns.

Imaging windows 15 may be formed of a single layer, or multiple layers, of material. For example, as shown in FIG. 1C, an imaging window of a substrate is formed of two layers 46 and 48. Top layer 48 may comprise, for example, a silicon oxide-based material, e.g., SiO_{z}, where z can have a wide range based upon the substrate specification, particle beam used and biochemistry processes of an application (e.g., z=1.5, 2.0, or 2.5). The top layer may optionally be a material compatible with biological synthesis/assembly processes. The top layer may be optionally doped, e.g., to mimic different types of glass used in common chemistry. Bottom layer 46 may comprise, for example, a silicon nitride-based material, e.g., SiₓN_{y}, where x and y can have a wide range based upon the substrate specification, particle beam and biochemistry processes of an application. In some embodiments, x and/or y can range from 1-4, e.g., Si₃N₄. The bottom layer can be doped in some instances; however, in other instances, the bottom layer is undoped. The imaging window may optionally include additional layers of material(s) and/or different materials. Support portion 24 may comprise a silicon layer, and/or another material layer.

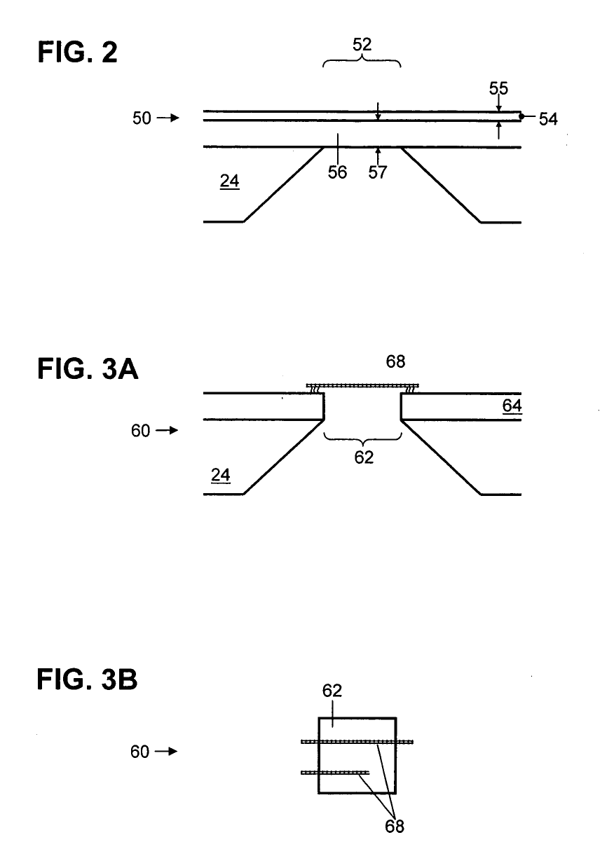

FIG. 2 shows another example of a portion of a substrate according to an embodiment of the invention. A substrate 50 includes an imaging window 52 having a top layer 54 (e.g., a SiO_{z} layer), which may have a thickness 55 of, for example, less than or equal to 20 nanometers, less than or equal to 10 nanometers, less than or equal to 5 nanometers, or less than or equal to 3 nanometers. The imaging window also includes a bottom layer 56 (e.g., a SiₓN_{y} layer), which may have a thickness 57 of, for example, less than or equal to 50 nanometers, less than or equal to 30 nanometers, less than or equal to 20 nanometers, less than or equal to 10 nanometers, or less than or equal to 5 nanometers. The combined thickness of layers 54 and 56 may be, for example, less than or equal to 60 nanometers, less than or equal to 40 nanometers, less than or equal to 30 nanometers, less than or equal to 15 nanometers, less than or equal to 10 nanometers, or less than or equal to 5 nanometers. In some cases, the combined thickness of layers 54 and 56 and the materials used to form the layers are chosen such that the imaging window is substantially optically transparent to electrons. For instance, upon binding of a binding partner to a ligand at the imaging window, exposure of the bound species to a particle beam, and imaging of the bound species, a contrast ratio of greater than 1.05:1, greater than 1:10, or greater than 1.2:1 may be produced. Of course, higher contrast ratios such as 2:1, 5:1, 10:1, or higher are also possible. Substrate 50 also includes substrate portion 24 (e.g., a Si layer), which may have a thickness of, for example, greater than 50 microns, greater than 100 microns, greater than 200 microns, greater than 500 microns, or greater than 1 mm. In some cases, the imaging window and/or other portions of the substrate can be formed of a single layer of material (e.g., SiO_{z}, which may be doped or undoped).

It should be understood that the substrate may include any number of layers, including a single layer, of material(s). For example, a substrate may be formed entirely of a silicon oxide-based material (e.g., SiO₂), such as a glass slide.

FIGS. 3A and 3B show another example of a portion of a substrate according to another embodiment of the invention. A substrate 60 includes an imaging window 62, which is a void and does not have any material layers. The substrate also includes support portion 24 (e.g., a Si layer), which may have various thicknesses. Optionally, the support portion may include one or more layers 64 (e.g., SiO_{z}), which may, for example, facilitate attachment of a chemical and/or biological species, and/or be compatible with biological synthesis/assembly processes. Ligands 68 (or probes) may be attached to a portion adjacent an imaging window (e.g., on layer 64 over a support portion 24), and may span a part, or all, of imaging window 62. As described in more detail below, in some cases, one or more ligands or probes may be bound to an attaching member (e.g., a nanoparticle) that may span a part, or all, of the imaging window. FIG. 3B shows a top view of imaging window 62 of substrate 60. The area of imaging window 62 may vary depending on, for example, the particular application, as well as the type and/or size of ligand 68. Advantageously, imaging windows having voids do not have materials that can interfere with a particle beam, which can result in an increased contrast ratio.

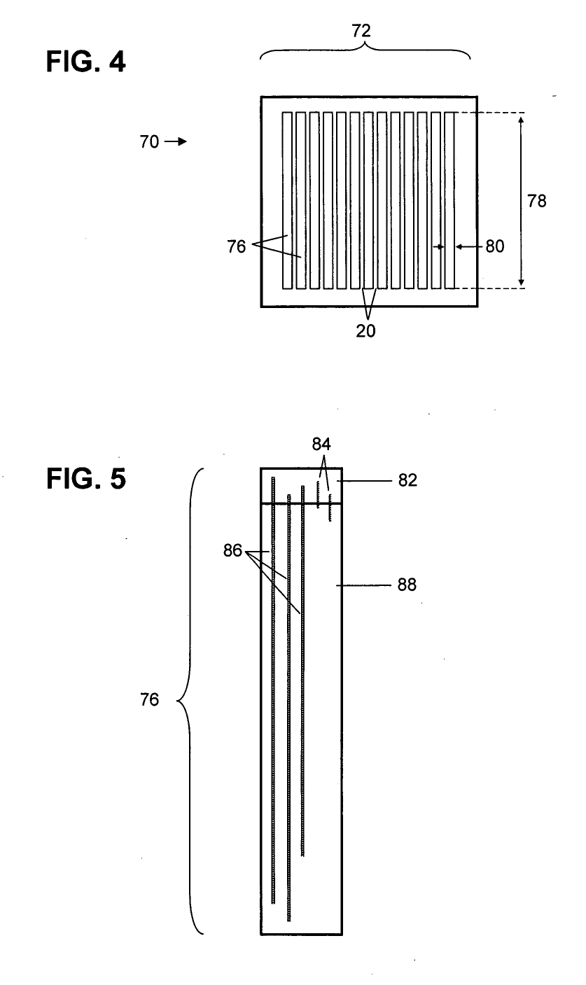

As discussed in more detail below, imaging windows can have a variety of shapes and/or sizes, which may depend on the particular application. For example, as shown in the embodiment illustrated in FIG. 4, a substrate 70 may include a frame 72 having a set of imaging windows 76 which may be rectangular and may have a ratio of length 78 to width 80 of greater than or equal to 3, greater than or equal to 7, greater than or equal to 10, greater than or equal to 15, greater than or equal to 20, or greater than or equal to 50. As described in more detail below, such dimensions of imaging windows may be particularly useful for directional molecular alignment (e.g., molecular combing) techniques. For example, as shown in the embodiment illustrated in FIG. 5, imaging window 76 includes an attachment region 82 to which are attached (e.g., covalently or non-covalently) ligand-binding partner pairs 86 after the ligands in the imaging window have bound (e.g., hybridized) with a binding partner. In some cases, unhybridized probes 84 are also present within the imaging window. Ligand-binding partner pairs 86 can be spread out along the length of the imaging window into alignment region 88 using directional molecular alignment techniques. Suitable alignment techniques have been described, for example, in U.S. Pat. No. 6,303,296 or International Patent Publication No. WO 95/21939, which are incorporated herein by reference. Directional molecular alignment techniques can minimize the overlap of the ligands to facilitate imaging. Other shapes and arrangements of imaging windows and species within imaging windows are also possible and described in more detail below. For instance, in some embodiments, probes and/or ligand-binding partner pairs can be attached to a part of the substrate outside of the imaging window, while a portion of the probe and/or ligand-binding partner pair is within the imaging window.

Suitable substrates may include a material having a rigid or semi-rigid surface. In some embodiments, at least one surface of the substrate is substantially flat, although in other embodiments, the substrate may have topographies such as raised regions, etched trenches (e.g., which can function as flow channels), surface roughness, or the like. Substrates may also be porous in some embodiments. The substrate may be biological, non biological, organic, inorganic, or a combination of any of these, existing as, for example, a planar or non-planar surface, sheet, slide, wafer, bead, web, fiber, tube, capillary, microfluidic channel, reservoir, strand, precipitate, gel, sphere, container, capillary, pad, slice, film, plate, or other structure. The substrate may have any convenient shape, such as a disc, square, sphere, circle, etc. In some embodiments, the substrate is preferably flat but may take on a variety of alternative surface configurations. For example, in some cases, the substrate may include raised or depressed regions on which synthesis/assembly takes place (e.g., for attaching a ligand). The substrate and its surface preferably may form a rigid support on which to carry out the reactions described herein. In some cases, surfaces of substrates can be tailored by certain treatments (e.g., with chemicals to form a thin film). In some cases, at least one portion or component of the substrate is removable.

It should be understood that other substrates may also be suitable.

In some embodiments, at least a portion of the substrate is formed of a material that does not substantially interfere with a particle beam (e.g., electron beam) that can be used for imaging a sample on a substrate as described herein. In other embodiments, a substrate has one or more voids where no substrate material exists. Materials and configurations of substrates may also be chosen based on, e.g., transparency to the wavelength of light used for lithographic assembly techniques.

In certain embodiments, a substrate is formed of one or more layers that may comprise, for example, a silicon oxide-based material, e.g., SiO_{z}, where z can have a wide range based upon the substrate specification, particle beam and biochemistry processes of an application, a silicon nitride-based material (e.g., SiₓN_{y}, where x and y can have a wide range based upon the substrate specification, particle beam and biochemistry processes of an application), carbon, aluminum oxide (A₂O₃), boron nitride, and/or other materials known to those of ordinary skill in the art as effectively transparent to electron imaging beams. The substrate and its surface may also be chosen to provide appropriate electronic characteristics, in addition or as an alternative to, appropriate optical characteristics. Other substrate materials will be readily apparent to those of skill in the art upon review of this disclosure. In addition, it is expected that different materials will afford different optimum thicknesses, with consideration given to compatibility with lithography, chemical reactions, durability, costs, and reproducibility.

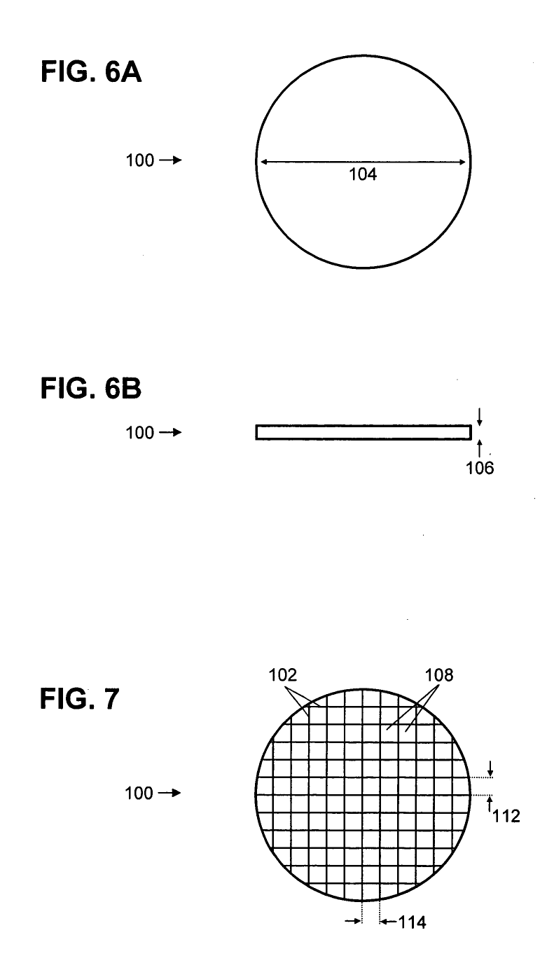

FIGS. 6A and 6B show top and side views, respectively, of a wafer (e.g., a silicon wafer), all or a portion of which can be used as a substrate of the invention. Wafer 100 may have any suitable size, such as, for example, a diameter 104 of 2 inches, 4 inches, 6 inches, 8 inches, or greater. Wafer 100 may also have any suitable thickness 106 such as, for example, less than or equal to 1 millimeter, less than or equal to 500 microns, less than or equal to 250 microns, less than or equal to 100 microns, or less than or equal to 50 microns. Although FIG. 6A shows a round wafer, the wafer can have any suitable shape and may include, e.g., a 1 inch by 3 inch slide.

As shown in the embodiment illustrated in FIG. 7, wafer 100 may be separated along lines 102 to form multiple substrates 108. Such separation may take place either before or after linkers or features have been patterned onto the substrate. Substrates 108 may have any suitable length 112 or width 114. For example, the length and/or width of the substrate may be less than or equal to 25 centimeters, less than or equal 10 centimeters, less than or equal to 1 centimeter, less than or equal to 7 millimeters, less than or equal to 3 millimeters, less than or equal to 1 millimeter, or less than or equal to 500 microns.

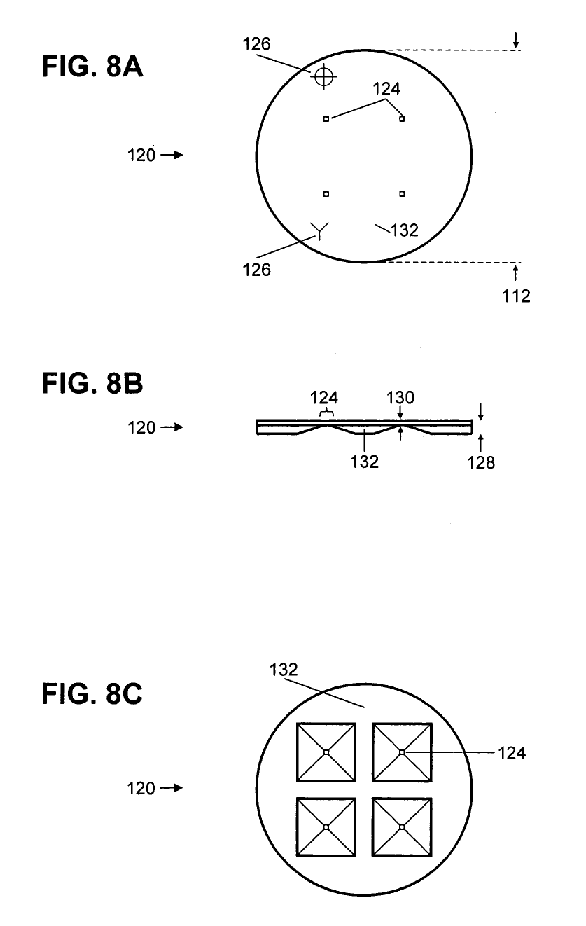

FIGS. 8A-8C show a top, side, and back view of substrate 120 according to one embodiment of the invention. As shown in the illustrative embodiment, substrate 120 includes a plurality of frames 124, each of which may include one or more imaging windows. The substrate may also include one or more registration marks 126, which may aid in the alignment of the substrate with an instrument. Substrate 120 may have more than one layers of thickness as shown in FIG. 8B. In one embodiment, thick portion 128 of the substrate is defined by the thickness of the wafer from which the substrate was formed, and thin portion 130 of the substrate may be defined by the thickness of frame 124. Frame 124 may be formed of one or more material layers and, in some embodiments, can include more than one levels of thickness. As shown, the frames are supported by support portions 132.

The substrate may have distinct regions such as imaging windows that can be fabricated using, for example, standard tools of lithography to give rise to a nano-scaled array. Such fabrication can be performed on a thin film that is later transferred to a support structure, or onto a thin film that has already had a supporting structure attached. The lithographic steps may be performed with a supporting mechanism to give mechanical strength to the film. This can be a temporary support that is later removed, or a permanent support that is an integral part of the final product.

According to some embodiments, one or more surfaces of the substrate are etched using well known techniques to provide for desired surface features.

The substrate, an imaging window of a substrate, and areas around or between imaging windows, can be of any suitable size or shape. For example, squares, ellipsoids, rectangles, triangles, circles, or portions thereof, along with irregular geometric shapes, may be utilized. Duplicate assembly areas may also be applied to a single substrate for purposes of redundancy.

In some embodiments, a single substrate supports one or more ligands, more than about 10 different ligands (e.g., monomer sequences), and preferably more than about 100 different ligands, although in other embodiments, more than about 10³, 10⁴, 10⁵, 10⁶, 10⁷, or 10⁸, different ligands are provided on a substrate. These ligands may be positioned, for example, on one or more imaging windows. In some embodiments, within a region of the substrate in which a ligand such as a polymer sequence is synthesized, the polymer sequence is substantially pure (i.e., the polymer has the desired sequence). However, in other embodiments, polymer sequences that are not substantially pure may be used, for example, polymer sequences that are not complete as designed due to prematurely stopping the synthesis of the polymer, or because a step was skipped during synthesis such that the polymer has the wrong sequence. In some embodiments, the density of ligands on a substrate can be adjusted to allow for individual binding partners to bind and to subsequently be analyzed with minimal interaction or interference with nearby molecules.

According to some embodiments, several ligands are intentionally provided within a feature so as to provide an initial screening for biological activity, after which materials within regions exhibiting significant binding are further processed and/or evaluated.

In other embodiments, an array of ligands of the desired dimensions and properties is prepared on a substrate other than the final substrate and is then transferred to the final substrate. This transfer can be performed by stamping, or by bringing one surface into proximity of another surface. In one embodiment, transfer is performed with photo-labile or electro-labile connectors to the original substrate that are severed via exposure to energy beams when brought into proximity or contact with the final substrate. In preferred embodiments, the final substrate would have been prepared with active molecules that include ligands or linkers that match a corresponding molecule on the terminus of the ligands of the original substrate.

As described above, an imaging window may be defined by an area of a substrate having a total thickness of, for example, less than or equal to 60 nanometers, less than or equal to 40 nanometers, less than or equal to 30 nanometers, less than or equal to 20 nanometers, less than or equal to 10 nanometers, less than or equal to 5 nanometers, less than or equal to 3 nanometers, or less than or equal to 1 nanometer. An imaging window may be formed of a material type that minimizes interference with a specialized variant of a particle beam being used for the application including material described above. The material of an imaging window may also be chosen with consideration to its transparency to the wavelength of light used in a lithographic assembly method. For example, in one embodiment, at least a portion of an imaging window is a thin film (having one or more layers) that is relatively optically transparent to electron beams of energies typical of electron microscopes (e.g., greater than 10, 50, 100, 200 or 300 kV). Each material layer(s) of the imaging window may have a thickness of, for example, less than or equal to 60 nanometers, less than or equal to 40 nanometers, less than or equal to 30 nanometers, less than or equal to 20 nanometers, less than or equal to 10 nanometers, less than or equal to 5 nanometers, less than or equal to 3 nanometers, or less than or equal to 1 nanometer. In some cases, the thinnest imaging window that does not create practical problems from fragility during processing, use, storage, etc. is preferred. For example, in its most preferred embodiment, the imaging window material may create no interference with a particle beam passing therethrough, and, therefore, can allow the clearest analysis of the sample. In some embodiments, an imaging window is a void in a substrate that does not include any material layers.

A substrate may include any suitable number of imaging windows and may depend on a particular application. For instance, a substrate may include greater than or equal to 1, greater than 10, greater than 10², greater than 10⁴, greater than 10⁵, or greater than 10⁶ imaging windows.

On any given substrate, the imaging windows can be of different sizes, shapes, and distances from one another. The shape of the imaging window may vary by application including, but not limited to, circular, rectangular or other shapes. The smoothness of the comers and edges may also vary. In some instances, at least one imaging window of a substrate spans an area of less than or equal to 10 square millimeters, less than or equal to 5 square millimeters, less than or equal to 1 square millimeters, less than or equal to 500 square microns, less than or equal to 250 square microns, less than or equal to 100 square microns, less than or equal to 50 square microns, less than or equal to 10 square microns, less than or equal to 1 square micron, less than or equal to 500 square nanometers, or less than or equal to 300 square nanometers.

The distance between the centers of adjacent imaging windows (i.e., the window pitch) may vary depending on the particular application. For example, the window pitch may be less than or equal to 7 millimeters, less than or equal to 5 millimeters, less than or equal to 100 microns, less than or equal to 20 microns, less than or equal to 7 microns, or less than or equal to 2 microns.

For each application of the invention, a different tradeoff may be made between imaging window material, thickness and surface area to optimize the economics. Designs having frames with more than two thicknesses (e.g., as shown in the embodiment illustrated in FIG. 1B) may be produced to maximize the surface area for imaging windows and arrays while maintaining structural strength with consideration given to compatibility with lithography, chemical reactions, durability, costs, and reproducibility.

In general, thinner imaging windows may have smaller surface areas and/or wider divider portions to manage fragility. Designing thinner imaging windows with wider divider portions may result in fewer features per array and, therefore, higher cost per feature.

The number of imaging windows on a substrate, the number of arrays on a substrate, and the surface coverage per array may vary by application and required economics. In one embodiment, a single array spans one imaging window. In another embodiment, a single array spans a plurality of imaging windows. In yet other embodiments, a single array spans more than one frame. Multiple arrays can also be present within a single imaging window, multiple imaging windows, a single frame, or multiple frames.

Imaging windows can be manufactured using common techniques in the semiconductor industry by those of ordinary skill in the art. For instance, thinning of a wafer to create an imaging window may be accomplished by lithographic shaping and etching or other techniques using light, masks, electron beams, chemicals, other methods or a combination of several methods.

In most embodiments, thinning will generally take place on one side of the substrate to allow a uniform and smooth surface on the other side for the feature building process. However, in other embodiments, thinning may occur on more than one sides of a substrate.

One or more separate materials with desired surface properties may be deposited on a wafer before the thinning process so that the material(s) can become the material on which the features are attached. Common methods of material deposition may be used including, but not limited to, chemical vapor deposition, plasma enhanced chemical vapor deposition, or other deposition techniques that result in suitable surface properties.

In some cases, an imaging window is manufactured as a thin film that is later transferred to a substrate support structure. The lithographic shaping methods may be performed with a supporting mechanism to give mechanical strength to the thin film, but a temporary support that is later removed could also be used.

As described above and as shown in FIGS. 1-3 and 8, substrates described herein may have multiple levels of thickness to allow more imaging windows to be included on the substrate, while providing stability for synthesis/assembly of features, as well as subsequent handling with samples. For example, a wafer with thickness of hundreds of microns may be thinned inside the substrate perimeter to a few or tens of microns to maintain a strong outer edge structure. Other relatively thick internal structural elements may be left before the final thin imaging windows are created. Thick structural elements may reduce the usable imaging window surface areas; therefore, the substrate design may include a balance of these trade-offs while meeting the application requirements.

In some embodiments, thinning of a substrate to form an imaging window may be partially completed before the linkers and/or ligands are added, and then finished subsequently to achieve the target thickness using methods such as vapor etching, which will not damage the attached linkers and/or ligands. This approach can allow the imaging window to be thicker, stronger and/or more resistant to fracture during the linker/ligand attachment process. For example, in one embodiment, a substrate may be thinned along its back side to create a well or channel in the substrate, and an imaging window along one side of the substrate (e.g., as shown in embodiment illustrated in FIG. 2). The well or channel may be subsequently filled (partially or completely) with a filler material (e.g., PMMA) to form a thicker imaging window portion. Linkers, ligands, and/or probes may then be synthesized and/or attached to the front side of the imaging window. The filler material can then be washed out of the well or channel, leaving behind the thin imaging window with linkers, ligands, and/or probes attached to its front side.

The imaging window may have markings that can be imaged by a particle beam instrument or another instrument indicating the position of the imaging window and/or an array, an X/Y orientation, or other reference indicators to facilitate the management and interpretation of images.

A substrate can include a variety of different configurations of imaging windows. For example, in one embodiment, all imaging windows of the substrate are part of a single array and the same sample can be applied to the whole substrate surface, or to the parts of the substrate where the imaging windows include features. In another method, a substrate includes more than one array and all features in any imaging window are part of the same array.

In some cases, the width of the substrate portions between imaging windows on a flat surface (the divider portions) are chosen at least in part according to the particular method of subjecting the imaging windows to a sample so as to prevent cross-contamination of other arrays on the substrate when the sample is applied. In such cases, non-mechanical fluid deposition and spreading controls may be used. For example, drop size, humidity and/or temperature may be controlled such that a sample does not spread to an adjacent array. In some instances, a surface between adjacent arrays can be treated to control spreading of the sample (e.g., by adding a hydrophobic film, a wetting agent, and/or by embedding electrical conductors in the substrate and creating an electrical field to control spreading). In another embodiment, one or more of the following factors are used to prevent cross-contamination of sample between at least a first and a second array on the substrate when the sample is applied to one of the arrays: structures on top of or cut into the surface, the width and/or thickness of the divider portions, thickness of the thicker substrate portion, mechanical fluid deposition and spreading controls, non-mechanical fluid deposition and spreading controls, or other factors. In some cases, elevated walls (that may optionally be hydrophobic) may be carved into the substrate or built up separately. For instance, in one embodiment, the walls may be part of the silicon oxide and/or silicon nitride-based material. In another embodiment, the walls may be formed of resist material, which can be exposed and/or washed away when desired.

In certain embodiments, an imaging window includes a single array. In other embodiments, an imaging window can include greater than 1, greater than 10, greater than 50, greater than 500, greater than 1,000, greater than 5000, or greater than 10,000 arrays.

In some embodiments, a frame (e.g., including one or several imaging windows) and/or the region upon which several distinct ligands are synthesized, has an area of, for example, less than 3 square centimeters, less than 100 square millimeters, less than 10 square millimeters, or less than 1 square millimeter. In other embodiments, the area may be less than 500 square microns, less than 100 square microns, less than 10 square microns, less than 4 square microns, or less than 1 square microns. In certain embodiments, the area of a frame may be less than 500,000 square nanometers, less than 250,000 square nanometers, less than 100,000 square nanometers, less than 10,000 square nanometers, less than 1,000 square nanometers, or less than 500 square nanometers. In some embodiments, a ligand or binding site within an imaging window may have an area such as one described above, or in other embodiments, may be as small as a single molecule.

Layout of features to form an array in one or more imaging windows may depend on the particular application. For example, in one embodiment, location of each feature composed of specific ligands can be used to identify the binding partners that bind to that feature for applications like gene expression. Multiple features in an array can include the same ligands (e.g., the same composition of monomers) to measure high quantities of binding partners, to provide redundancy, or for other reasons. In some cases, features can be designed and added for control purposes. For example, ligands can be designed so they should not bind with any binding partners in a sample. Such a control may be useful for determining false positives, e.g., any binding to that feature would be false and indicate a problem with one or more procedures. Another type of control would be ligands designed to bind only with reference standard ligands introduced into the sample.

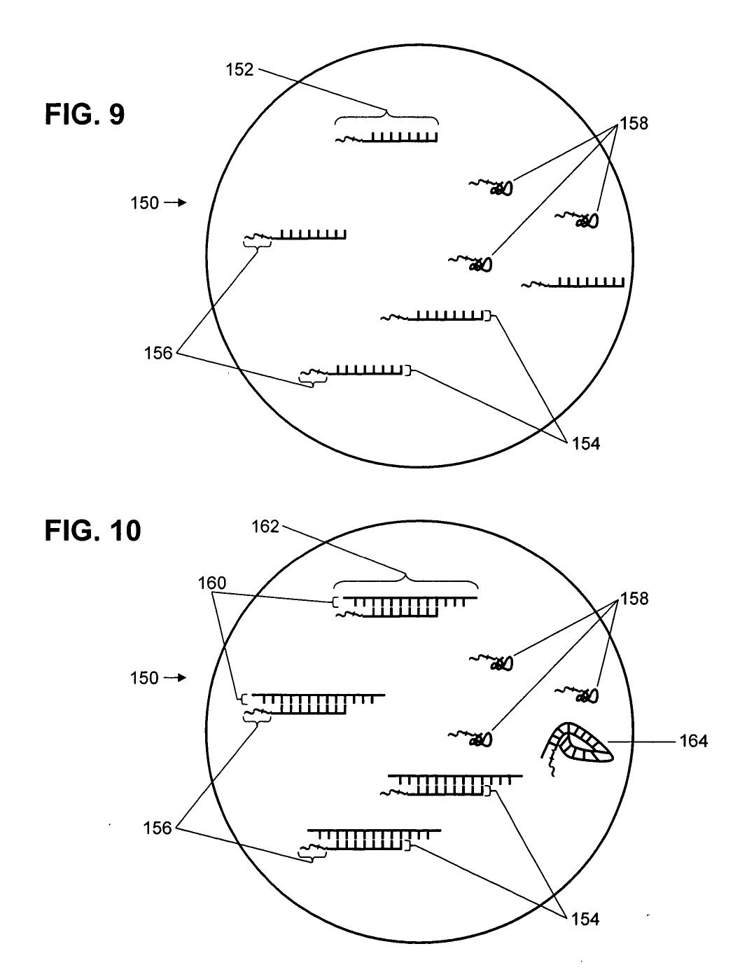

FIG. 9 shows an example of a top view of a feature prior to binding with a binding partner according to one embodiment of the invention. Feature 150 may be positioned on an imaging window of a substrate and may include one or more ligands 154, which may be optionally attached (e.g., covalently) to a surface of the imaging window via linkers 156 to form probe 152. In some embodiments, some or all of the ligands in a feature are not attached to the surface via linkers. In some cases, ligands 154 are single stranded DNA (ssDNA). Depending on the length or design of the ligands, certain probes 158 may be tangled after being positioned on the surface.

FIG. 10 shows an example of a top view of feature 150 after binding (e.g., hybridization) of ligands 154 with binding partners 160 according to one embodiment of the invention. Although a binding partner may have a similar length as a ligand in some embodiments, it should be understood that in many instances, only a portion of a binding partner may be bound to a ligand. As such, portions of the binding partner that are not bound to the ligand may curl up and/or have a tangled configuration. As shown, ligands 154 are relatively spread out potentially due to a stronger structure of the pairing with binding partners 160. In some cases, however, a tangled, unhybridized probe 158 may be in the same feature and may not be bound with a binding partner 160. In other cases, there may be a looped ligand-binding partner pair 164 due to, for example, conditions of the surface, the length of the molecule, inconsistencies in the wet chemistry process, or other reasons.

It should be understood that any suitable ligand can be used with substrates described herein. For instance, in some embodiments, ligands 154 are proteins, antibodies, antigens, modified DNA strands, or combinations thereof. In one particular embodiment, a ligand is multivalent (i.e., it has more than one active site) and can bind with more than one binding partner. For example, a ligand may be an avidin molecule, which has four active sites for binding with biotin. Upon exposure of the avidin molecule to biotin, an avidin-biotin pair may be formed. If the biotin molecule is modified to have another ligand attached thereto, the modified biotin molecule can now act as a ligand for another binding partner. Accordingly, multiple levels of binding can occur and molecules may have multiple roles as both ligands and binding partners.

In another embodiment, a ligand such as modified single (or double) stranded DNA can have a side chain (e.g., a linker) to which a molecule is attached (e.g., avidin) and either or both the DNA and the molecule can act as ligands for different binding partners. It yet another embodiment, a ligand such as a single stranded DNA may have a looped configuration such that it is attached to a surface at or near the middle of the polymer. The DNA may have a first sequence on one side of the polymer for binding with a first binding partner, and a second sequence on the other side of the polymer for binding with a second binding partner. Other variations of ligands are also possible.

In some embodiments, the number of features in an array may be small, for example, an array may include 1, 2, less than or equal to 5, less than or equal to 10, less than or equal to 20, less than or equal to 40, less than or equal to 70, or less than or equal to 100 features. A relatively small number of features in an array may be designed, for instance, for certain applications that seek fast processing including, but not limited to, identification of certain binding partners (e.g., biotoxin), clinical diagnostics, genotyping or fingerprinting. In other embodiments, the number of features can be large, for example, greater than or equal to 100, greater than or equal to 200, greater than or equal to 500, greater than or equal to 1,000, greater than or equal to 10,000, greater than or equal to 100,000, greater than or equal to 500,000, or greater than or equal to 1,000,000 features. A large number of features may be useful for some applications including, but not limited to, whole genome expression profiling or genetic sequencing.

In some embodiments, a single imaging window includes one or a plurality of features. For example, a single imaging window may include greater than or equal to 1, greater than or equal to 5, greater than or equal to 10, greater than or equal to 50, greater than or equal to 100, greater than or equal to 500, greater than or equal to 1,000, greater than or equal to 5,000, greater than or equal to 10,000, greater than or equal to 100,000, greater than or equal to 200,000, greater than or equal to 500,000, greater than or equal to 1,000,000 features. In other embodiments, such numbers of features may be spread out over more than one imaging window.

Locations of the features on a substrate may include positional identification for some applications. In certain other applications, e.g., de novo sequencing or other types of genetic analysis, locations of features on the substrate do not include positional identification.

In certain embodiments, a substrate that can be used with a particle beam instrument includes greater than or equal to 2, greater than or equal to 10, greater than or equal to 25, greater than or equal to 50, greater than or equal to 100, greater than or equal to 500, greater than or equal to 1,000, greater than or equal to 5,000, or greater than or equal to 10,000 arrays. In one particular embodiment, every array on such a substrate is defined by features in a subset of the imaging windows, and all the features in an imaging window are part of the same array. In another embodiment, one or more imaging windows of such a substrate include more than one array. The features belonging to each array within the imaging window are located contiguously and identified by a positional location. In such substrate variations, each imaging window may include, for example, greater than or equal to 1, greater than or equal to 2, greater than or equal to 10, greater than or equal to 100, greater than or equal to 1,000, greater than or equal to 5,000, greater than or equal to 10,000, greater than or equal to 50,000, greater than or equal to 100,000, greater than or equal to 500,000, greater than or equal to 1,000,000 features. Each feature may include, for example, less than or equal to 1,000,000, less than or equal to 500,000, less than or equal to 100,000, less than or equal to 50,000, less than or equal to 10,000, less than or equal to 5,000, less than or equal to 1,000, less than or equal to 100, less than or equal to 10, less than or equal to 2, or less than or equal to 1 linkers and/or ligands attached to a surface of an imaging window.

In some embodiments, linker molecules are provided on a substrate. In one particular embodiment, a terminal end of a linker molecule is provided with a reactive functional group protected with a protective group, which is removable by exposure to either photons or electrons (e.g., photolithography or electron beam lithography). Using photo-lithographic or electron beam lithographic shaping methods, the removable protective group can be exposed to light or an electron beam. This can result in removal of the protective group from the linker molecules in selected regions. The substrate can then be contacted with a first monomer (or ligand) that reacts with exposed finctional groups on the linker molecules. In one embodiment, the monomer is an amino acid containing a photoremovable or electron beam removable protective group at its amino or carboxy terminus and the linker molecule terminates in an amino or carboxy acid group bearing a photoremovable or electron-beam removable protective group.

In some cases, a second set of selected regions is, thereafter, exposed to the light or electron beam and the removable protective group on the linker molecule/protected amino acid is removed at the second set of regions. The substrate is then contacted with a second monomer (or ligand) which may attach a photoremovable or electron-beam-removable protective group for reaction with exposed functional groups. This process can be repeated to selectively apply monomers until polymers of a desired length and desired chemical sequence are obtained. Electro-labile or photo-labile groups can then be optionally removed and the sequence may be optionally capped. Side chain protective groups, if present, can also be removed.

It is to be understood that the descriptions herein are intended to be illustrative and not restrictive. Many embodiments will be apparent to those of skill in the art upon reviewing the above description. By way of example, although aspects have been described primarily with reference to the use of photoremovable protective groups, it will be readily recognized by those of ordinary skill in the art that sources of radiation other than light could also be used. For example, in some embodiments it may be desirable to use protective groups which are sensitive to electron beam irradiation, x-ray irradiation, in combination with electron beam lithograph, or x-ray lithography techniques. Alternatively, a protective group could be removed by exposure to an electric current or an acidic environment possibly created by chemical reaction when exposed to photons.

In one embodiment, regions on a substrate (e.g., a frame or an imaging window) can be assembled to have a mixture of different polymers built up in the region. In other embodiments, the regions have differing sizes and/or shapes to facilitate manipulation of molecules that bind to the sites. In other embodiments, the regions have differing concentrations of polymers to facilitate imaging of subsequently bound and labeled species without them clumping so much as to make imaging impractical.

In another embodiment, different regions are processed to have distinct reactive sites at the terminus of the polymer. Then, a mixture of reagents can be added such that each type of terminal reactive site reacts with a specific reagent, allowing for the addition of different monomers at different locations at the same time.

In some embodiments, one or more ligands are attached (e.g., covalently or non-covalently) to a surface of a substrate without the use of linkers.

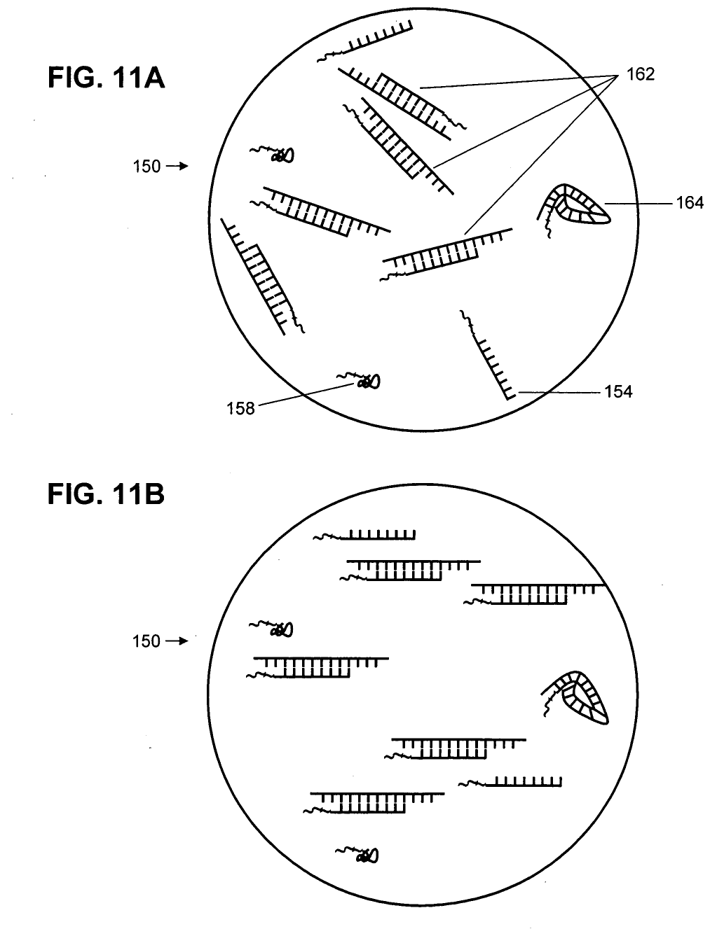

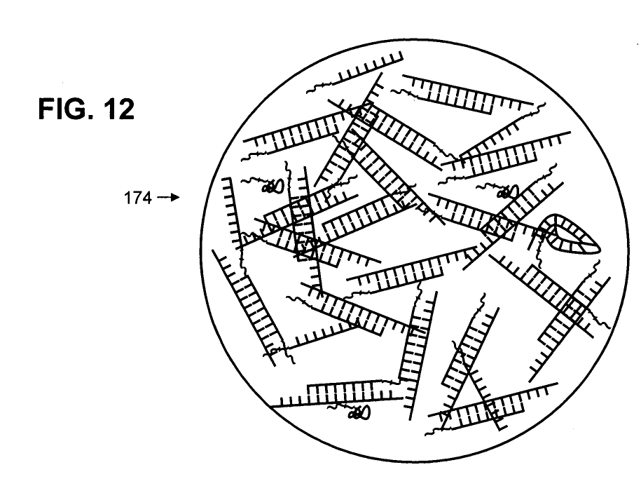

A substrate may include any suitable number of ligands or probes for detecting a binding partner. As described above, due to the high sensitivity of the methods of detection described herein, each feature of a substrate can be made up of a fewer number of ligands or probes than that of certain microarrays. For instance, in certain embodiments, a feature may have less than 10⁵, less than 10⁴, less than 10³, less than 500, less than 100, less than 10, or 1 ligand(s) or probe(s) per square micron. FIG. 11A shows an example of a feature, as viewed from above, having ligand-binding partner pairs with relatively low density and degree of overlap. FIG. 11B shows the same feature as FIG. 11A but after using a directional molecular alignment method. FIG. 12 shows an example of a feature, as viewed from above, having ligand-binding partner pairs with relatively high density and degree of overlap without being subjected to a directional molecular alignment method.

In some embodiments, ligands and/or probes are positioned on a substrate (e.g., at an imaging window) indirectly via one or more attaching members. For example, a ligand may be attached (e.g., covalently or non-covalently) to an attaching member, and at least a portion of the attaching member may be positioned on an imaging window. Attaching members may be biological, non biological, organic, inorganic, or a combination of any of these. Non-limiting examples of attaching members include linkers, particles, nanoparticles, nanotubes, quantum dots, planar or non-planar surfaces, sheets, slides, wafers, beads, webs, fibers, tubes, capillaries, microfluidic channels, reservoirs, strands, precipitates, gels, spheres, containers, pads, slices, films, plates, or other structures. Attaching members may have any suitable shape and/or size.

Several methods can be used for fabricating features (e.g., including linkers, ligands, and/or probes) on imaging windows. In some embodiments, lithographic assembly methods are used. In one particular embodiment, a lithographic assembly method includes the use of an optical lithography instrument, exposure of the substrate through masks, a protective method involving photo-resists and attaching complete ligands to each feature. In such a method, a substrate surface can be cleaned and prepared. Cleaning may be performed with, for example, organic solvents, methylene chloride, dimethylformamide (DMF), ethyl alcohol, or the like. In some embodiments, preparation is performed with substances such as silanes that promote adhesion for attachment of linkers to the substrate surface. For example, one end of a linker may include a silane group, and another end group of the linker may include an aldehyde and/or amino group, which can be used for attaching prefabricated DNA or in situ synthesis of oligonucleotides. Linkers may be attached to a surface as individual molecules, as a monolayer, as incomplete layers of molecules, or as multi-layers of molecules. A photo-resist layer can be applied to the surface of the imaging windows and, in some embodiments, to the whole substrate surface. An optical lithography instrument and masks can be used to expose all specific areas in the imaging windows where features will be located. The exposed photo-resist can then be washed away to leave the prepared surface. Additional preparation steps may be used at this time to promote linker attachment.

Linkers in a solution can be applied to imaging windows and maintained in an environment that promotes attachment of one end of the linkers to the surface of the imaging window. The other end of the linker may be designed to bind with any suitable ligand in the application, and may involve a photo labile protective method. The environment (e.g., acidity levels, temperature levels, period of duration or other factors) may be compatible with the linkers in use. Linker attachment may be controlled to achieve a density on the surface that is designed for the ligands and application of the array. In some embodiments, linkers and their attached ligands are positioned as close together as possible while still allowing the data analysis module to discriminate the samples in a subsequent step of the chosen application. Control can be achieved through dilution in deliberate proportion with water or other fluids, mixing linkers with particles that will evenly diffuse the linkers in solution, or using another controllable technique that will position linkers on the surface for the attachment process at intervals that achieve the targeted density. After linker attachment is completed, the solution and any unattached linkers can be removed.

A ligand may be bound to the end of the linker using multiple cycles of light exposure through masks (e.g., photo-resist protective methods). One such method involves attaching complete ligands, e.g., an oligonucleotide, of selected length and sequence to the end of each linker in a feature. The first cycle can include applying a photo-resist protective method to all features, and then using optical lithography with masks to expose all features with the same ligand design. The exposed photo-resist can then be removed by washing (or using some other method), thereby leaving open feature areas on the substrate surface. Light exposure can also de-protect the linker ends. The complete ligands can then be applied in a solution to imaging windows and maintained in an environment that promotes attachment to the linkers in the open feature locations. Unattached ligands can then be removed and photo-resist may be applied to protect these feature areas. This de-protection, ligand attachment and protection cycle can be repeated until all features are completed. In some embodiments, the attachment of complete ligands may be preferred method for fabricating features, e.g., for certain arrays having a relatively low number of features, or for features including long ligands.

Another lithographic assembly method for fabricating features may include the use of an optical lithography instrument, exposure of the substrate through masks, a protective method involving photo-resists and building the ligand on the end of each linker with common DNA synthesis methods. The first cycle may include using optical lithography with masks to expose all features which start with a particular monomer in its ligand sequence. The exposed photo-resist can then be removed by washing, thereby leaving open feature areas on the substrate surface. Light exposure can also de-protect the linker ends. The particular monomers, with one end deactivated by a photo-labile protective method, can then be applied in a solution to imaging windows and maintained in an environment that promotes attachment to the linkers in the open feature locations. Unattached monomers can then be removed and photo-resist can be applied to protect these feature areas. This de-protection, monomer attachment and protection cycle can be repeated for a different monomer, adding one to either the linker or the previous monomer in every feature where that new monomer is next in the ligand sequence. These cycles can be repeated until all ligands and features are completed. When a ligand is completed, a capping molecule may then be added to the end. This DNA synthesis approach may be used for arrays with thousands of features or relatively short ligands.

In another embodiment, a method for fabricating features includes the use of an optical lithography instrument, exposure of the substrate without masks, a protective method involving photo-resists and attaching complete ligands to each feature. Several lithographic assembly methods exist and are in development that can focus light or other beams in millimeter, micron or nanometer dimensions on specific locations without the use of masks (i.e., “maskless lithography”). Such devices include, but are not limited to, spatial light modulators, digital light processing and other technologies to focus light or other beams on a surface.

In such a method, a substrate surface can be cleaned and prepared and can be optionally treated with substances such as silanes that promote adhesion for attachment of linkers to the substrate surface. Linkers may be attached to a surface as individual molecules, as a monolayer, as incomplete layers of molecules, or as multi-layers of molecules. A photo-resist layer can be applied to the surfaces of the imaging windows and, in some embodiments, to the whole substrate surface. A maskless optical lithography instrument can be used to expose the specific areas in the imaging windows where features will be located. The exposed photo-resist can then washed away to leave the prepared surface. Additional preparation steps may be used at this time to promote linker attachment.

Linkers in a solution can be applied to imaging windows and maintained in an environment that promotes attachment of one end of the linkers to the surface of the imaging window. The other end of the linker may be designed to bind with any suitable ligand in the application, and may involve a photo labile protective method. The environment (e.g., acidity levels, temperature levels, period of duration or other factors) may be compatible with the linkers in use. Linker attachment may be controlled to achieve a density on the surface that is designed for the ligands and application of the array. In some embodiments, linkers and their attached ligands are positioned as close together as possible while still allowing the data analysis module to discriminate the samples in a subsequent step of the chosen application. Control can be achieved through dilution in deliberate proportion with water or other fluids, mixing linkers with particles that will evenly diffuse the linkers in solution, or using another controllable technique that will position linkers on the surface for the attachment process at intervals that achieve the targeted density. After linker attachment is completed, the solution and any unattached linkers can be removed.

A ligand may be bound to the end of the linker using multiple cycles of maskless optical lithography and photo-resist protective methods. One such method involves attaching complete ligands, e.g., an oligonucleotide, of selected length and sequence to the end of each linker in a feature. The first cycle includes using maskless optical lithography to expose all features with the same ligand design. The exposed photo-resist can then be removed by washing or some other method, thereby leaving open feature areas on the substrate. Light exposure can also de-protect the linker ends. The complete ligands can then be applied in a solution to imaging windows and maintained in an environment that promotes attachment to the linkers in the open feature locations. Unattached ligands can then be removed and photo-resist may be applied to protect these feature areas. This de-protection, ligand attachment and protection cycle can be repeated until all features are completed.

Another method includes the use of an optical lithography instrument, exposure of the substrate without masks, a protective method involving photo-resists and building the ligand on the end of each linker with common DNA synthesis methods. The first cycle includes the use of maskless optical lithography to expose all features which start with a particular monomer in its ligand sequence. The exposed photo-resist can then be removed by washing or some other method, thereby leaving open feature areas on the substrate surface. Light exposure can also de-protect the linker ends. The particular monomers, with one end deactivated by a photo-labile protective method, can be applied in a solution to imaging windows and maintained in an environment that promotes attachment to the linkers in the open feature locations. Unattached monomers can then be removed and photo-resist may be applied to protect these feature areas. This de-protection, monomer attachment and protection cycle can be repeated for a different monomer, adding one to either the linker or the previous monomer in every feature where that new monomer is next in the ligand sequence. These cycles can be repeated until all ligands and features are completed. When a ligand is completed, a capping molecule may be added to the end.

In another embodiment, a method for fabricating features includes the use of an optical lithography instrument, exposure of the substrate through masks, and a protective method involving photo-labile protecting groups. In such a method, a substrate surface can be cleaned and prepared. Cleaning may be performed with, for example, organic solvents, methylene chloride, dimethylformamide (DMF), ethyl alcohol, or the like. In some embodiments, preparation is performed with substances such as silanes that promote adhesion for attachment of linkers to the substrate surface. Linkers may be attached to a surface as individual molecules, as a monolayer, as incomplete layers of molecules, or as multi-layers of molecules. A photo-resist layer can be applied to the surface of the imaging windows and, in some embodiments, the whole substrate surface. An optical lithography instrument and masks can be used to expose all specific areas in the imaging windows where features will be located. The exposed photo-resist can then be washed away to leave the prepared surface. Additional preparation steps may be used at this time to promote linker attachment.

Linkers in a solution can be applied to imaging windows and maintained in an environment that promotes attachment of one end of the linkers to the surface of the imaging window, as described above. After linker attachment is completed, the solution and any unattached linkers can be removed. The photo-resist can also be removed from the substrate. A ligand can be bound to an end of the linkers using multiple cycles of a light exposure through masks, and using photo-labile protecting group protective methods. One such method involves attaching complete ligands, e.g., an oligonucleotide, of selected length and sequence to the end of each linker in a feature. The first cycle includes applying a photo-resist protective method to expose all features with the same ligand design. The exposure of photo-labile protecting groups can leave open feature areas on the substrate surface. Light exposure can also de-protect the linker ends. The complete ligands can then be applied in a solution to imaging windows and maintained in an environment that promotes attachment to the linkers in the open feature locations. Unattached ligands can then be removed and photo-resist may be applied to protect these feature areas. This de-protection, ligand attachment and protection cycle can be repeated until all features are completed.

Another method is to build the ligand on the end of each linker with common DNA synthesis methods and using photo-labile protecting groups.

In another embodiment, a method for fabricating features includes the use of an optical lithography instrument, exposure of the substrate without masks, and a protective method involving photo-labile protecting groups. In one embodiment, a ligand can be bound to an end of a linker on a substrate using multiple cycles of a maskless optical lithographic assembly method and a photo-labile protective group protective method. In another embodiment, a ligand can be built on the end of each linker with common DNA synthesis methods.

In yet another embodiment, a method for fabricating features includes spotting complete ligands (or probes) onto a substrate, e.g., using an ink jet printed or another suitable device.

In some embodiments, the above methods for manufacturing features may be used with method variations. For instance, one method variation may include an array of features of the desired dimensions and properties that is prepared on an attaching member or substrate other than the final substrate, which is then transferred to the final substrate. This transfer can be performed by stamping, bringing into proximity, self-assembly, or by another other suitable method. In one embodiment, transfer is performed with photo-labile or electro-labile connectors to an attaching member, and are severed via exposure to energy beams when brought into proximity or contact with the substrate. In some embodiments, prior to transfer, the substrate can be prepared with active sites that include binding partners or linkers that match a corresponding linker on the terminus of the active regions of the attaching member.

In some instances, a complete ligand can be bound to a linker (e.g., to form a probe) before the linker/probe is attached to the substrate surface. In one embodiment, a complete ligand can be bound to a surface (e.g., covalently or non-covalently) without using a linker. In some instances, the complete ligand can be bound to an attaching member, and the attaching member may be bound (e.g., covalently or non-covalently) to a surface of the substrate.

Another method variation includes the use of a linker and/or ligand that is not bound at their respective ends. One or both could bind (e.g., to a surface or to another species) at some other place along the length of the polymer.

In some embodiments, the data analysis module may differentiate a ligand-binding partner pair from an unlabeled ligand by the shape, duplex width, and/or some other characteristic. Mismatched hybridization between binding partners and ligands can also be identified by shape, duplex width, and/or another characteristic. For example, binding of a binding partner along the full length of a ligand can be differentiated from binding that takes place along only a portion of the ligand. In other embodiments, the use of one or more labels on both ligands and binding partners may be advantageous in some applications. In yet other embodiments, the ligands are labeled, the binding partners are not labeled, and the data analysis module may differentiate a ligand-binding partner pair from the ligand by the shape, duplex width, and/or some other characteristic. The binding partner may be, for example, MRNA or cDNA, which may benefit from less sample pre-processing. Of course, in other cases, a binding partner is labeled and the ligand is not labeled.

In some cases, the initial feature opening steps using a photo-resist protective method prior to linker attachment can be performed with a different protective method.

Lithographic assembly methods described herein may, in some embodiments, involve the use of an electron beam. The types of protective methods, e.g., optical-resist in some methods, and photo-labile groups in other methods, can be changed to work with this electron beam lithographic assembly method.

In some embodiments, directional molecular alignment can be performed across the arrays, either after ligands have been synthesized, or after ligands have bound with binding partners to form ligand-binding partner pairs. This alignment method can increase the straightness of the ligands and the ability to discriminate them separately.

Many biological samples are essentially transparent to electron microscopy and related techniques at resolutions adequate for molecular imaging. Consequently, appropriate labeling may be necessary to allow for imaging when electron microscopy is used for identification, quantification, sequencing, fingerprinting, and mapping (e.g., optical mapping) of polymers. Any suitable label that can provide contrast between a bound sample and a substrate can be used in combination with embodiments described herein. In some embodiments, a suitable label includes a contrast atom label, which can interfere with a beam from a particle beam instrument, and can provide a suitable contrast ratio between the label and the surrounding material and substrate. For instance, in one embodiment, a contrast atom label has a higher atomic number than atoms that are normally found in the material being analyzed at that location in the molecular structure. A contrast atom label may, in some embodiments, include atoms of relatively high atomic number that do not otherwise impede the biochemistry, physical chemistry, physics and electronic requirements of the detection process. In one embodiment, labels are incorporated covalently into nucleic acids by using modified dNTP's or dNTP analogues in a nucleic acid amplification, transcription, or reverse-transcription process. In another embodiment, binding factors of sufficient electron opacity can be imaged without labels. In still another embodiment, cofactors, binding proteins or any sort of recognition reagent can be modified to include one or more atoms of sufficient atomic number (singly or in aggregate) to allow for detection via electron microscopy and the like.

In certain embodiments, labels can be in the form of dyes. For example, intercalating species that intercalate only with double-stranded nucleic acids can be used rather than modified dNTP's, so long as the intercalating species also has properties that make it detectable using a particle beam instrument.

In other embodiments, labels can be formed from short-chain (e.g., five-mer, ten-mer, twenty-mer) nucleotide chains of known sequence and with unique collections of high atomic number atoms. These labels of known sequence are useful for hybridizing to polymers that have been immobilized on an array by virtue of a known terminal sequence that matches the sequence of the region. The labels can then be washed over the array, allowing the identification of distinct sequences within a region. This may be the case with alleles of a certain gene that all anneal to the same region but that consequently bind to different labels, creating a region of heterogeneous labels.

In some cases, labels described in related International Publication No.: WO 2006/019903, which is incorporated herein by reference in its entirety, can be used.

With an appropriate label selected, the detection system best adapted for high resolution and high sensitivity detection may be selected. As indicated above, an electron-optics detectable system, e.g., scanning electron microscope or transmission electron microscope, either with or without additional instruments may be used with substrates described herein.

In some embodiments, detection of a binding event includes the use of a heavy-atom(s). In some instances, the chemical structure of a ligand or binding partner is modified with one or more heavy-atoms, which can be detected with a particle beam instrument. For example, in one embodiment, a heavy-atom-modified nucleotide is used to detect interactions with oligonucleotide probes of known sequence.

The enablement of a sequencing process by hybridization is based, at least in part, upon the ability to synthesize a large number of the possible overlapping sequence segments of a target biological molecule, distinguish those probes which hybridize with fidelity from those which have mismatched bases, and to analyze a highly complex pattern of hybridization results to determine the overlap regions. In one embodiment, detection of the positions which bind the target sequence can be through electron-interacting labels on the target. Although a single-atom label may be used, other sorts of labels, e.g., multiple heavy atoms, tri-flouro-methyl groups, atoms replacing typical nucleotide atoms, multiple heavy atoms connected via linkers, small diffracting ligands, etc. may also be used. Because the oligonucleotide probes on an array may be positionally defined, the location of a hybridized entity can directly translate to the sequences which hybridize. Thus, analysis of the positions provides a collection of subsequences found within the target sequence. These subsequences can be matched with respect to their overlaps so as to assemble an intact target sequence.

In other embodiments, labels are not required in order to determine binding on a substrate.

In some embodiments, detection of binding on a substrate can be performed using a particle beam instrument. The substrate may have a large variety of ligands at known regions (e.g., features) on a surface thereof. In some cases, the substrate is exposed to an electron-interacting labeled binding partner which binds to one or more of the ligands. The substrate can be placed in a microscope detection apparatus for identification of regions where binding takes place. The microscope detection apparatus may include, for example, a monochromatic or polychromatic electron source for directing high-energy electrons at the substrate, an instrument for detecting transmitted, reflected and/or deflected electrons from the substrate, and an instrument for determining the locations and patterns of the electrons. The electrons may be emitted at, e.g., greater than or equal to 10 kV, greater than or equal to 50 kV, greater than or equal to 100 kV, greater than or equal to 200 kV, or greater than or equal to 300 kV. The instrument for detecting electrons may include, for example, one or more digital cameras in conjunction with one or more phosphor imaging plates. The instrument may include a moveable stage to mechanically position the area on the substrate under the particle beam and it may also include the ability to shift the particle beam to take multiple images without moving the substrate or the stage. The instrument may also include a camera for rapidly locating a region of the substrate for detailed imaging. Some embodiments will include multiple cameras that collect data simultaneously or in sequence from different sections of the imaged substrate. An instrument for determining a region from the images on the substrate may include, for example, an x/y translation table for the substrate. Translation of the substrate regions and data collection can be recorded and managed by an appropriately programmed digital computer.

As described herein, substrates of the invention can be useful for de novo sequencing of an unknown polymer sequence, for sequencing of a partially unknown polymer sequence, for verifying known sequences, for identifying small differences from known sequences, for quantization of nucleic acids or proteins (commonly called expression), for molecular diagnostics, for fingerprinting polymers, for mapping homologous segments within a sequence, for assessment of methylation status of nucleic acids, for “seeing” DNA roll-up in a histone, for “seeing” RNA loop through a ribosome, for polymer identification and for general screening of specific interactions. In some cases, articles and methods described herein can be used for optical mapping using relatively low resolution TEM. It should be understood, however, that the substrates and arrays described herein can be applied to determining other polymers, e.g., carbohydrates, polypeptides, hydrocarbon synthetic polymers, and the like. For determining non-polynucleotides, the sequence specific ligands may be antibodies (modified or otherwise) specific for a particular subunit sequence.

By reducing the number of chemical manipulations required, reducing the number of sample molecules required, measuring smaller differences in quantization, reducing the required dimensions, and/or automating most of the steps, the speed, accuracy, and/or reliability of these procedures can be greatly enhanced. By reducing the absolute size of the substrate, as well as the array components, substantially greater number of units can be produced per production run resulting in a lower cost per unit. In some cases, maskless lithographic assembly methods can increase flexibility and reduce cost for custom feature design by eliminating the fixed cost of mask production. Moreover, photo-labile protective methods can reduce error rates in the production process.

Automation of sequencing manipulations is also provided. Automation of substrate production, scanning, and analysis steps can minimize the need for human intervention. This can simplify the tasks and promote reproducibility.

In one embodiment, a substrate described herein is designed for gene expression. The substrate can be manufactured from a silicon nitride wafer having a thickness of, for example, 300-400 microns. The substrates may optionally be square with rounded corners and may measure approximately 3 millimeters edge to edge. A 4-inch diameter wafer can, therefore, produce approximately 750 substrates. Fabrication steps to create the substrates, imaging windows, and features in arrays can be performed simultaneously on every substrate. Each substrate may have, for example, three thicknesses. The first step may be to use lithographic shaping methods to chemically etch an area, e.g., 100 by 100 microns, in the middle of each substrate to a thickness of less than or equal to 30 nanometers. The edges of the individual substrates on the wafer also can be scored with thin lines where they can be separated in a later fabrication step. The next fabrication step may be to use chemical etching to create approximately 16,900 imaging windows in the middle of the 100 by 100 micron area. Each imaging window can measure, e.g., 500 by 500 nanometers with a window pitch of, for example, 750 nanometers between adjacent imaging windows. Each imaging window can be thinned to a layer of silicon dioxide deposited to a thickness of, e.g., 2-5 nanometers, on top of silicon nitride (e.g., 10-20 nanometers thick). The chemical etching and scoring steps may use optical lithographic shaping and photo-resist methods, substances and instruments that are familiar to those of ordinary skill in the art.

In some embodiments, there is one feature in each imaging window on the substrate, and, therefore, the whole substrate is one array. Each feature includes a different oligonucleotide ligand designed to hybridize with single strand cDNA fragment binding partners that are complementary copies of mRNA for common tissues that have undergone reverse transcription.

In certain embodiments, all ligand sequences in the features are about 25 oligomers in length and are bound at one end to linkers of about 5 monomers in length. When linkers and oligomer ligands are attached at their respective ends, the combination may be approximately 10 nanometers in length. To minimize the overlap of ligand-binding partner pairs after hybridization and washing, the substrate may be designed such that each pair has approximately 400 square nanometers of target spacing around each linker attachment point (e.g., so the pair can lie in any direction). Given each feature may be approximately 500 by 500 nanometers in size, a feature can accommodate approximately 625 ligand-binding partner pairs spaced randomly.

Surfaces of the imaging windows of the substrate can be washed and prepared for linker attachment. Photo-resist and maskless optical lithographic assembly methods can be used to expose the feature locations and the exposed resist can be washed away to leave open feature areas on the surface of the imaging windows. Next, linkers with a photo-labile protecting group on one end can be attached within each feature area. That end of the linker may be designed to bind with any oligonucleotide after de-protection. The linkers can be applied in a solution designed to produce attachments with the targeted spacing. This solution may be applied to all imaging windows and held in an environment that promotes attachment of linkers touching the surface in each feature. Unattached linkers and solution can then be washed away, as well as the remaining photo-resist. The preceding methods, materials and instruments are known to those of ordinary skill in the art. Next, one feature production cycle can be completed. Maskless optical lithographic assembly methods can be used to expose the one or more feature locations where a specific ligand will be attached to the substrate. The exposure can release the photo-labile protecting groups on all linkers in those features. A solution with many copies of complete specific ligands (e.g., 25-mer oligonucleotides of a specific sequence with a photo-labile protecting group on one end) can be placed on the arrays with pipette, inkjet instrument or another microfluidic instrument. The wafer can be held in an environment designed to promote binding of the linker and ligand ends that have no protective groups. The unattached ligands can then be washed away. Additional feature production cycles may then be completed for every type of feature until all arrays on all substrates are complete.

Using the method described above, approximately 750 microarrays can be manufactured on the wafer. The microarrays can then be separated by physically separating the wafer along the substrate edge lines scored earlier.

In another embodiment, a substrate for gene expression is designed to include 2-micron round features with a 3 micron pitch between features. Features may include 60 base ligands attached to a surface of an imaging window with 20 monomer linkers spread throughout the whole feature area. The linker density may be designed such that a 60 base ligand on a 20 monomer linker avoids overlap of ligand-binding partner combinations (e.g., DNA double strands).

In one embodiment, a sample of MRNA can be converted to cDNA through reverse transcription to become the binding partners. Labels may be incorporated on, for example, all or a portion of the thymine and cytosine monomers of the cDNA using a polymerase chain reaction (PCR) process. Other methods for incorporating labels are also possible. The labels may be medium molecular weight atom(s) designed to be incorporated in the structure of one type of monomer, for example, such that the thymine has one type of label and the cytosine has another. The labeled sample binding partner can then be placed on the array and held in an environment (e.g., with appropriate temperature, pH, humidity etc.) that promotes hybridization to complementary ligands where they are present on the array. Unbound sample cDNA can then be washed away. The reverse transcription, PCR labeling and hybridization steps are known to those of ordinary skill in the art. The label design and chemistry is further described in related International Publication No: W02006/019903. In some cases, any monomers of the single strand binding partners that are not hybridized to the ligands are cut off prior to washing away the unbound sample cDNA.

The substrate with the attached labeled binding partner can then be inserted in a low resolution transmission electron microscope of, e.g., approximately 120 kV accelerating voltage, which can focus on one or more features to create a clear image on a phosphor screen. This image can be captured in one or more shots by a digital camera designed to work with the TEM. The TEM staging mechanism can then center the camera on an adjacent feature and another image can be taken with the camera. This cycle can be repeated until all the features are imaged. These TEM imaging steps are known to those of ordinary skill in the art.

Images can be processed by modified machine vision or some other software in a data analysis module. The labels of the binding partner are designed to interfere with a low-resolution transmission electron microscope beam and may appear as a relatively high contrast dark string in a feature that can be discriminated from the low contrast surrounding material. The oligonucleotide ligands with no labeled binding partner attached are typically made of low contrast DNA material that does not stand out from other background noise in the TEM image. Each dark string in a feature image can, therefore, indicate the presence of a single labeled binding partner that can be counted by the data analysis module. The count of dark strings may indicate the level of gene expression and the cDNA type can be identified by the specific location of the feature in the array. When the array is manufactured, the location of each feature can be associated with a unique type of cDNA that can preferentially hybridize with the ligands inside that feature based upon the designed ligand monomer sequence.

In another embodiment, a substrate described herein is designed for genetic re-sequencing. The substrate can be manufactured from a silicon nitride wafer having a thickness of, for example, 300-400 microns. The substrates may optionally be square with rounded corners and may measure approximately 3 millimeters edge to edge. A 4-inch diameter wafer can, therefore, produce approximately 750 substrates. Fabrication steps to create the substrates, imaging windows, and features in arrays can be performed simultaneously on every substrate. Each substrate may have three thicknesses. The first step may be to chemically etch an area, e.g., 100 by 100 microns, in the middle of each substrate to a thickness of less than or equal to 30 nanometers. The edges of the individual substrates on the wafer also can be scored with thin lines where they can be separated in a later fabrication step. The next fabrication step may be to use chemical etching to create approximately 850 imaging windows in the 100 by 100 micron area. Each imaging window can measure, e.g., 1000 by 7,000 nanometers with a window pitch of, for example, between 1,500 to 7,500 nanometers such that there is a dividing portion about 500 nanometers wide between adjacent imaging windows. Each imaging window can be thinned (e.g., by etching away portions of silicon and/or silicon nitride) and a layer of silicon dioxide having a thickness of, e.g., 2-5 nanometers, may be deposited. The chemical etching and scoring steps may use optical lithographic shaping and photo-resist methods, substances and instruments that are familiar to those of ordinary skill in the art.

In some embodiments, there is one feature in each imaging window on the substrate, and, therefore, the whole substrate is one array. Each feature includes a different oligonucleotide ligand designed to hybridize with single strand cDNA fragment binding partners that are complementary copies of mRNA for common tissues that have undergone reverse transcription. The oligonucleotide ligand in each feature may be a sequence chosen to hybridize with a single strand section on the end of an approximately 20,000 monomer duplex strand binding partner being targeted.

In certain embodiments, all ligand sequences in the features are about 25 oligomers in length and are bound at one or more positions to linkers of about 5 monomers in length. If linkers and oligomer ligands are attached at their respective ends, the combination may be approximately 10 nanometers in length. For the genetic re-sequencing application, the substrate may be designed such that the targeted spacing is the amount where 25-50 linker and ligand combinations are dispersed within an attachment region area of, e.g., 1000 by 200 nanometers. Every attachment region may be positioned at the same end of the imaging window filling the 1000 nanometer dimension and extending down 200 nanometers into the 7,000 nanometer window dimension. The rest of the imaging window forms an alignment region, where the 20,000 monomer ligand-binding partner pair can be spread out using directional molecular alignment techniques after hybridization.

Surfaces of the imaging windows of the substrate can be washed and prepared for linker attachment. Photo-resist and maskless optical lithographic assembly methods can be used to expose the feature locations and the exposed resist can be washed away to leave open feature areas on the surface of the imaging windows. Next, linkers with a photo-labile protecting group on one end can be attached within each feature area. That end of the linker may be designed to bind with any oligonucleotide after de-protection. The linkers can be applied in a solution designed to produce attachments with the targeted spacing. This solution may be applied to all imaging windows and held in an environment that promotes attachment of linkers touching the surface in each feature. Unattached linkers and solution can then be washed away, as well as the remaining photo-resist. The preceding methods, materials and instruments are known to those of ordinary skill in the art. Next, one feature production cycle can be completed. Maskless optical lithographic assembly methods can be used to expose the one or more feature locations where a specific ligand will be attached to the substrate. The exposure can release the photo-labile protecting groups on all linkers in those features. A solution with many copies of complete specific ligands (e.g., 25-mer oligonucleotides of a specific sequence with a photo-labile protecting group on one end) can be placed on the arrays with pipette, inkjet instrument or another microfluidic instrument. The wafer can be held in an environment designed to promote binding of the linker and ligand ends that have no protective groups. The unattached ligands can then be washed away. Additional feature production cycles may then be completed for every type of feature until all arrays on all substrates are complete.

Using the method described above, approximately 750 microarrays can be manufactured on the wafer. The microarrays can then be separated by physical separating the wafer along the substrate edge lines scored earlier.

The next steps in a genetic re-sequencing embodiment may be to prepare the sample, hybridize it to the microarray, take images and process them. For instance, double stranded DNA can be cut with enzymes at positions that start with the target sequence of a designed ligand and end after, for example, approximately 25-60 bases. The double stranded DNA may be heated or otherwise separated to obtain single stranded DNA and DNA strands complementary to the single stands can be produced by any nucleic acid replication method known to the skilled artisan, such as PCR. A PCR process can be used to produce (and optionally amplify) a double stranded DNA molecule by extension of a primer after hybridization of the primer to the single stranded DNA molecules. The primer can be, for example, a 25-mer sequence complementary to the ligand. Labels can be incorporated into the newly formed complementary single stranded DNA molecules or double stranded DNA molecules during the PCR process by including labels on, for example, all thymine and cytosine monomers used in PCR. These labeled DNA molecules become the binding partners.

The labels may be medium molecular weight atom(s) designed to be incorporated in the structure of one type of monomer, for example, such that the thymine has one type of label and the cytosine has another. The labeled sample binding partner can then be placed on the array and held in an environment (e.g., with appropriate temperature, pH, etc.) that promotes hybridization to complementary ligands where they are present on the array. Unbound sample cDNA can then be washed away. A directional molecular alignment technique can then be used across all imaging windows to spread the bound entities into the alignment region of the imaging window where they can covalently bind to the surface and straighten out the helix as the meniscus passes. The goal is to minimize the overlap of the bound entities to facilitate imaging. The reverse transcription, cDNA cutting, PCR transcription, PCR labeling, hybridization steps and directional molecular alignment are known to those of ordinary skill in the art. The label design and chemistry is further described in related International Publication No: WO2006/019903.

The substrate with the attached labeled binding partners can then be inserted in a high-resolution TEM of which can focus on a part of an imaging window to create a clear contrast ratio image on a phosphor screen. This image can be captured in one or more shots by a digital camera designed to work with the TEM. The TEM staging mechanism can then center the camera on an adjacent portion of the imaging window or frame, and another image or series of images can be taken with the camera. Additionally and/or alternatively, the beam may be shifted to change the portion being imaged by the camera. This cycle can be repeated until each imaging window and all the imaging windows are imaged. These TEM imaging steps are known to those of ordinary skill in the art.

Images can be processed by modified machine vision or some other software in a data analysis module. With a high resolution TEM, the different labels of the binding partners can be identified. The labels may appear as a relatively high contrast string of different types of beads that can be discriminated from the low contrast surrounding material. The oligonucleotide ligands with no labeled binding partner attached are typically made of low contrast DNA material that does not stand out from other background noise in the TEM image. Each spot in an image related to a label identifies the specific monomer the label was attached to. In this example, since thymine and cytosine have labels, the unlabeled monomers that uniquely bind in the DNA double helix (adenine and guanine, respectively) can also be identified by the data analysis module. In other embodiments, all four monomers will be labeled differently. A sequence of approximately 20,000 monomers on the binding partner can be read. Multiple copies of the binding partner (e.g., 25-50) attached to the feature can allow quality control of the reading. When the array is manufactured, the location of each feature can be associated with a unique fragment of cDNA whose end can preferentially hybridize with the ligands inside that feature based upon the designed ligand monomer sequence. The identification of the ligand by feature in the array can facilitate the computer informatics task of assembling a whole genetic sequence. The image processing methodologies are described in related International Publication No.: WO 2006/019903.

In another embodiment, a substrate described herein is designed for de novo genetic sequencing. De novo genetic sequencing can be performed with the same methods as genetic re-sequencing (described above) except for the ligand-binding partner pair design. The linker end in each feature that is not bound to the surface may be designed to be universal and may not have a ligand attached thereto; in other words, it can be designed to bind with the end of any binding partner or any molecule, e.g., a sample cDNA single strand that has been turned into a double strand through PCR and labeled on one or both strands. More complex computer informatics may be required to identify duplicate binding partner sequences and reassemble different sequences of, e.g., up to 50,000 monomer reads into the proper genetic sequence. Sequencing of mRNA is also possible.

In one example, RNA molecules can be bound to the substrate (to specific or non-specific ligands), followed by production of a complementary DNA strand using reverse transcribed PCR (RT-PCR). The RT-PCT process can be used to incorporate labels into the new complementary DNA strand as described elsewhere herein. This labeled RNA/DNA hybrid can then be subjected to directional molecular alignment and images of the labeled DNA captured using, for example, a TEM as is described elsewhere herein. Alternatively, after formation of the RNA/DNA hybrid molecule by RT-PCR, the DNA strand can be attached to a ligand in the same location, after which the RNA strand can be removed, for example by preferentially dissolving it using a hydroxide wash. Attachment of the DNA strand can be accomplished using the various methods described herein; it also can be facilitated by including in the PCR reaction mixture a nucleotide that can attach to ligands. A second labeled DNA strand, complementary to the first DNA strand, then can be produced using PCR. Directional alignment of the molecules and image capture then can proceed as described elsewhere herein. These methods can facilitate determining expression and sequence using the same sample.

In another embodiment, a substrate described herein is designed for DNA sequencing. For DNA sequencing, the feature area may be a rectangle measuring, e.g., 1 micron by 1 micron. The feature pitch may be created with, for example, a 1 micron space between feature areas. Within the feature area may be an attachment region at one end of the feature, which may measure the full 1 micron width and extend down the rectangle to about 0.2 microns. The remaining alignment region may be, for example, 1 micron by 0.8 microns to leave room for approximately 50 linkers (e.g., each of which may be spaced 20 nanometers apart) with ligand-binding partner pairs having about 2,000 bases in a DNA double strand. Strands having hundreds of thousands of bases can also be used. The spacing and number of the linkers may also vary.

In another embodiment, a substrate described herein is designed for molecular diagnostics. For molecular diagnostics, a feature area may, for example, be round and about 18 microns wide with a 20 micron feature pitch (e.g., much like those in some gene expression fluorescent systems). The linker density may be similar to that of the gene expression application. Since the goal of molecular diagnostics is to look for major differences in expression, the features may be large but may be imaged at less detail (e.g., many large features may be imaged in one shot showing degrees of opacity, but not individual molecules of the features). Individual molecule accuracy may be performed for some features of the hybridized substrate if desired.

In some cases, binding of a ligand to a binding partner is not based solely, or at least in part, on complementary base pairing. The polynucleotide targets, may, for example, be double or triple stranded in various applications. A triple stranded specific interaction may be sometimes desired, or a protein or other specific binding molecule may be utilized. For example, various promoter or DNA sequence-specific binding proteins might be used, including, e.g., restriction enzyme binding domains, other binding domains, and antibodies.

Thus, specific recognition reagents besides oligonucleotides may be utilized.

In some embodiments, a substrate described herein is designed for non-polynucleotide embodiments. In one embodiment, ligands described herein are polypeptides. These polypeptides may be protein binding domains from enzymes or other proteins which display specificity for binding. For example, an antibody molecule may be used, and monoclonal antibodies may be particularly desired. Classical methods may be applied for preparing antibodies, see, e.g., Harlow and Lane (1988) Antibodies: A Laboratory Manual Cold Spring Harbor Press, New York; and Goding (1986) Monoclonal Antibodies: Principles and Practice (2d Ed.) Academic Press, San Diego. Other suitable techniques for in vitro exposure of lymphocytes to the antigens or selection of libraries of antibody binding sites are described, e.g., in Huse et al. (1989) Science 246:1275-1281; and Ward et al. 91989) Nature 341:544-546, each of which is hereby incorporated herein by reference. Unusual antibody production methods are also described, e.g., in Hendricks et al. (1989) BioTechnology 7:1271-1274; and Hiatt et al. (1989) Nature 342:76-78, each of which is hereby incorporated herein by reference. Other molecules which may exhibit specific binding interaction may be useful for attachment to a VLSIPS™ technology substrate by various methods, including the caged biotin methods, see, e.g., Barrett et al. (1993) U.S. Pat. No. 5,252,743.

Antibody-specific reagents may be particularly useful for polypeptide, carbohydrate, and synthetic polymer applications. Individual specific reagents might be generated by an automated process to generate the number of reagents necessary to advantageously use a high density positional matrix pattern. In an alternative approach, a plurality of hybridoma cells may be screened for their ability to bind. A matrix possessing the desired sequences whose binding specificity may be used. Each cell might be individually grown up and its binding specificity determined by a suitable apparatus and technology. An alternative strategy would be to expose the same matrix to a polyclonal serum of high titer. By a successively large volume of serum and different antibodies, each region of the substrate can have attached thereto a substantial number of antibody molecules with specificity of binding. The substrate, with non-covalently bound antibodies, can be derivatized and the antibodies may be transferred to an adjacent second substrate in the matrix pattern in which the antibody molecules had attached to the first matrix. If the sensitivity of detection of binding interaction is sufficiently high, such a low efficiency transfer of antibody molecules may produce a sufficiently high signal to be useful for many purposes, including the sequencing applications.

In another embodiment, capillary forces may be used to transfer the selected reagents to a new matrix, to which the reagents would be positionally attached in the pattern of the recognized sequences. Or, the reagents could be transversely electrophoresed, magnetically transferred, or otherwise transported to a new substrate in their retained positional pattern.

In some embodiments, the binding partner need not actually be labeled if a means for detecting where interaction takes place is available. As described herein, for a nucleic acid embodiment, such may be provided by an intercalating dye which intercalates only into double stranded segments, e.g., where interaction occurs. See, e.g., Sheldon et al. U.S. Pat. No. 4,582,789.

In certain embodiments, the target sequence is homogeneous, both with respect to the total sequence and with respect to the ends of each molecule. Homogeneity with respect to sequence is important to avoid ambiguity. It is preferable that the target sequences of interest not be contaminated with a significant amount of labeled contaminating sequences. The extent of allowable contamination will depend on the sensitivity of the detection system and the inherent signal to noise of the system. Homogeneous contamination sequences may be particularly disruptive of the sequencing procedure.

Although certain target polynucleotides compatible with techniques described herein may have a unique sequence, the target molecules need not have identical ends. In fact, the homogeneous target molecule preparation may be randomly sheared to increase the numerical number of molecules. Since the total information content remains the same, the shearing may result in a higher number of distinct sequences which may be labeled and bound to the probe. This fragmentation may give a superior signal relative to a preparation of the target molecules having homogeneous ends. The signal for the hybridization may be dependent on the numerical frequency of the ligand-binding partner interactions. If a sequence is individually found on a larger number of separate molecules, a better signal may result. In fact, shearing a homogeneous preparation of the binding partner may often be preferred before the labeling procedure is performed, thereby producing a large number of labeling groups associated with each subsequence.

In another embodiment, substrates described herein can be designed to work with a self-assembly methodology. The feature areas on the substrate may not be identified with a specific ligand that is designed to bind with a complementary binding partner. When a solution containing sample binding partners contacts the substrate, the binding partners can self-assemble to binding points within one or more imaging windows, and may comprise a code identifying the specific type of binding partner. One example would be a method with sample binding partners attached to attaching members, e.g., nanoparticles, that self-assemble onto the substrate. In such an embodiment, the substrate may be designed such that the imaging windows are holes with a shape that allows a particular nanoparticle to partially penetrate the window in a three-dimensional orientation, but may not pass through the window. One variation would be a truncated conical nanoparticle where the narrower end of the cone penetrates a substrate imaging window hole in the substrate. The hole diameter may be smaller than the diameter of the larger end of the conical nanoparticle such that the nanoparticle can be positioned part-way, but not all the way, through the hole. The nanoparticles can self-assemble on the substrate and can be oriented with the same side of the nanoparticle facing away from the substrate. The nanoparticles may or may not have the same rotational orientation. Other shapes of imaging windows and/or attaching members can also be used.

In one embodiment, such a self-assembly method can allow genetic analyses, e.g., when the nanoparticle is substantially transparent to the particle beam used. Binding partners used with such methods can incorporate atoms (e.g., contrast atoms) as labels that produce a discemable contrast ratio or signal-to-noise ratio after imaging with the particle beam. These binding partners may be attached to the nanoparticles prior to self-assembly on the substrate. One method of attaching a binding partner to a nanoparticle includes functionalizing the surface(s) of the nanoparticle, possibly only on the surface designed to face away from the substrate, then putting the nanoparticles in solution with specific ligands in an environment to promote attachment (e.g., covalently or non-covalently). A code uniquely identifying the specific ligand can also be attached to each nanoparticle (preferably on the surface designed to face away from the substrate), e.g., by etching the nanoparticle, attaching identifiable dyes, or other methods. The code can be read by the particle beam image or another system so that the location of the nanoparticle on the substrate is associated with the specific ligand. Batches of these coded nanoparticles with attached ligands can be created for each target binding partner in the experiment. Measured amounts of each chosen type of coded nanoparticles can be introduced into a solution with the experimental sample binding partners to promote binding of ligand-binding partner pairs on the nanoparticle. After contact with the substrate and self-assembly is complete, the nanoparticle codes can be read and the particle beam may be exposed for imaging.

Different genetic analysis applications may use different particle beams and imaging resolutions. One example would produce relative light intensity measures to estimate volume of different binding partners in the experimental sample. The accuracy may be increased compared to current fluorescent technologies due to, at least in part, eliminating bias from high amplification and fluorescent tag attachment and measurement consistency. The same substrate design may be used for all experiments because the coded nanoparticles with ligand-binding partner pairs may self-assemble into any substrate hole.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention.

What is claimed is:

1. A substrate, comprising:
at least a first imaging window having a thickness of less than or equal to 30 nanometers; and
at least a first linker attached to a surface of the first imaging window.

2. A substrate as in claim 1, further comprising a ligand attached to the first linker.

3. A substrate as in claim 2, wherein the ligand is a nucleic acid polymer.

4. A substrate as in claim 1, further comprising more than one linker attached to the surface of the first imaging window.

5. A substrate as in claim 1, wherein the imaging window is rectangular with a ratio of length to width of greater than or equal to 7.

6. A substrate as in claim 1, further comprising a second imaging window having a thickness of less than or equal to 30 nanometers and at least a second linker on a surface of the second imaging window.

7. A substrate as in claim 6, wherein a pitch between the first and second imaging windows is less than or equal to 3 microns.

8. A substrate as in claim 6, wherein the first and second imaging windows are separated by a substrate portion having a thickness of greater than 30 nanometers.

9. A substrate as in claim 8, wherein the thickness of the substrate portion is between 31-200 nanometers.

10. A substrate as in claim 1, comprising a plurality of imaging windows.

11. A substrate as in claim 10, wherein the plurality of imaging windows span an area of less than or equal to 7 square millimeters.

12. A substrate as in claim 10, wherein the density of imaging windows is greater than or equal to 100 imaging windows per square centimeter.

13. A substrate as in claim 10, comprising a plurality of features on a surface of one or more imaging windows, each feature comprising ligands having the same composition of monomers.

14. A substrate as in claim 13, wherein the plurality of features defines an array.

15. A substrate as in claim 13, wherein at least one feature has an area of less than or equal to 3 square microns.

16. A substrate as in claim 13, wherein the density of features is greater than or equal to 100,000 features per square millimeter.

17. A substrate as in claim 13 wherein at least one feature comprises less than or equal to 500 linkers per μm².

18. A substrate as in claim 13, wherein a pitch between adjacent first and second features is less than or equal to 3 microns.

19. A substrate as in claim 13, further comprising first and second arrays, each array comprising more than one feature.

20. A substrate as in claim 19, wherein a substrate portion separating the first and second arrays has a thickness of greater than 200 microns.

21. A substrate as in claim 1, wherein the imaging window comprises at least a first and a second layer of materials.

22. A substrate as in claim 21, wherein the first, top layer is a silicon oxide-based layer and the second, bottom layer is a silicon nitride-based layer.

23. A substrate as in claim 1, wherein the imaging window is formed of a single layer of material.

24. A substrate as in claim 23, wherein the material is a silicon-oxide based material.

25. A substrate as in claim 1, wherein a monomer at an end of the linker is attached to a protecting group.

26. A substrate as in claim 25, wherein the protecting group is a photo-labile protecting group.

27. A substrate, comprising:
at least a first imaging window; and
at least a first ligand attached to a surface of the first imaging window, wherein the first imaging window is capable of producing a contrast ratio of greater than 1.05:1 upon exposure of the first imaging window to a particle beam and upon imaging of the first ligand.

28. A substrate as in claim 27, wherein the ligand is labeled with a single atom.

29. A substrate as in claim 27, wherein the ligand comprises an internal label.

30. A substrate as in claim 27, wherein the particle beam is an electron beam having an energy of greater than 10 kV.

31. A substrate as in claim 27, wherein binding is detectable by an electron microscope at the first imaging window.

32. A substrate as in claim 27, wherein at least a portion of the imaging window is free of a substrate material.

33. A substrate as in claim 27, wherein at least a portion of the imaging window has a thickness of less than or equal to 30 nanometers.

34. A substrate, comprising:
a first portion comprising a silicon oxide-based layer and a silicon nitride-based layer;
a second portion having a thickness greater than the first portion, the second portion comprising a silicon oxide-based layer, a silicon nitride-based layer, and a silicon layer; and
a first linker attached to a surface of the first portion of the substrate.

35. A substrate as in claim 34, further comprising a feature including a first ligand attached to the first linker.

36. A substrate as in claim 35, further comprising a feature including at least two different ligands.

37. A substrate as in claim 34, wherein the first portion is an imaging window.

38. A substrate as in claim 34, wherein the first portion is substantially optically transparent to electrons.

39. A substrate, comprising:
at least a first imaging window having a thickness of less than or equal to 30 nanometers; and
at least a first ligand attached to a surface of the first imaging window.

40. A substrate as in claim 39, wherein the ligand is attached to the surface via an attaching member.

41. A substrate as in claim 40, wherein the attaching member is a linker.

42. A substrate as in claim 40, wherein the attaching member is a nanoparticle.

43. A substrate as in claim 39, wherein the ligand is attached to the surface directly without the use of an attaching member.

44. A substrate as in claim 39, wherein the ligand is a nucleic acid polymer.

45. A substrate as in claim 39, comprising a plurality of features on a surface of the first imaging window, each feature comprising ligands having the same composition of monomers.
